(19) United States
(12) Patent Application Publication
(10) Pub. No.: US 2024/0185949 A1
(43) Pub. Date: Jun. 6, 2024

(54) SYSTEMS AND METHODS FOR V(D)J CELL CALLING BASED ON THE PRESENCE OF GENE EXPRESSION DATA

(71) Applicant: 10x Genomics, Inc., Pleasanton, CA (US)

(72) Inventor: Sreenath KRISHNAN, Fremont, CA (US)

(73) Assignee: 10x Genomics, Inc., Pleasanton, CA (US)

(21) Appl. No.: 18/529,987

(22) Filed: Dec. 5, 2023

Related U.S. Application Data
(60) Provisional application No. 63/430,589, filed on Dec. 6, 2022.

(51) Int. Cl. G16B 20/00; C12Q 1/6881; G16B 10/00; G16B 25/10; G16B 30/10; G16B 35/20

(52) U.S. Cl. CPC G16B 20/00; C12Q 1/6881; G16B 10/00; G16B 25/10; G16B 30/10; G16B 35/20

(57) ABSTRACT

A computer-implemented method is provided for identifying V(D)J cells from at least one data set including a plurality of sequence barcode records from a plurality of single cells. Embodiments of the method include analyzing nucleic acid sequences from nucleic acid sequencing data set(s), comprising comparing a plurality of barcode records in a V(D)J sequencing data set(s) to a plurality of barcode records in a gene expression (GEX) data set(s), to identify a plurality of single V(D)J cells from V(D)J sequencing data set(s), wherein each single V(D)J cell has one or more full length V(D)J contigs.

### CROSS REFERENCE TO RELATED APPLICATION

This application claims priority to U.S. provisional patent application Ser. No. 63/430,589 filed Dec. 6, 2022, the entire contents of which is incorporated herein by reference and relied upon.

### FIELD

This description is generally directed towards systems and methods for analyzing immune cell clonotype data generated using single- and multi-modal single cell nucleic acid sequencing technologies. More specifically, there is a need for systems and methods to for identifying one or more V(D)J cells from a data set containing sequence barcode records from a plurality of single cells. There is also a need for systems and methods to visualize and present immune cell clonotype data so that it is readily analyzed and interpreted by a user. Systems and methods to assess, visualize, and present these data for analysis and interpretation are useful and readily applied to data generated using non-droplet and droplet-based single cell nucleic acid sequencing technologies, array-based microwell- and nanowell-based single cell nucleic acid sequencing technologies, in situ sequencing technologies, and spatially indexed single cell technologies.

### BACKGROUND

The immune system recognizes and eliminates non-self threats through a complex and layered network of both innate and adaptive immune cells. Robust characterization of this response and characterization of V(D)J sequences has proven challenging to perform in a high-throughput fashion using flow cytometry, CyTOF, and similar assays. One approach to addressing these limitations is to utilize multi-modal single cell technologies, including without limitation droplet-based single cell techniques. Applications of these technologies include the analysis of T cells, B cells, and peripheral blood mononuclear cells, e.g., pre- and post-vaccination samples, e.g., from influenza vaccines or other vaccines (or of samples collected from individuals affected by diseases such as systemic lupus erythematosus and other autoimmune disorders, chronic viral infection, and acute/non-chronic viral infection), or T cells/B cells/PBMCs from individuals treated with a drug or biological molecule such as a checkpoint inhibitor, anti-cancer drug, monoclonal antibody, or antibody-drug conjugate. Importantly, these single cell assays allow users to learn the full and paired sequences of heterodimeric and extremely polymorphic immune cell receptors of adaptive lymphocytes, e.g., T cells and B cells, and to identify from which single cell (and its corresponding phenotype, genotype, and antigen specificity) a given immune receptor had originated. This relationship is masked or not directly observable using bulk DNA and RNA-based sequencing assays and is not captured in a cost-effective or high-throughput fashion in plate-based assays.

Using this framework, T cell and B cell responses can be identified and used to implement an immune cell (B cells/T cells/PBMCs) clonotyping algorithm that immune receptor lineages at scale by combining untargeted and targeted gene expression, full-length immune cell receptor sequencing, surface protein expression and/or antigen capture, in addition to tag-based and genetic demultiplexing.

Current analysis platforms purportedly assign D genes yet cannot assign them confidently. Moreover, D gene assignments are not guaranteed to be consistent across a clonotype. These assignments are made, even though they are not confident, as they generally allow one to better understand what happened during junction region rearrangement. However, given current limitations, that understanding is often incomplete. This weakness of assignment is a consequence, for example, of the biology: D genes are short, and junction regions can be heavily edited during somatic hypermutation (SHM) and through non-templated indels during V(D)J recombination. As such, it is currently possible that where a D gene is aligned to given transcript bases, it is not the right D gene, or that the transcript bases represent some other part of the genome (not a D gene at all), or even random bases that were created during formation of the junction region. Tis a need for systems and methods that can more accurately determine optimal D gene assignment and/or junction region structure.

In addition, current analysis platforms have filtering processes which are designed to remove artifacts and unreliable transcripts which likely do not reflect actual B cells and/or T cells. These filters unfortunately also can filter out legitimate clonotypes that have a shared chain. This results in reduced cell recovery via the discarding of valid clonotypes.

As such, there is a need for systems and methods that can more accurately identify actual sequence barcodes which are cell-associated, rather than false reads or artifacts. In particular, there is a need for filtering workflows which identify single V(D)J cells that have one or more full-length productive V(D)J contigs from a plurality of single cells. In non-limiting embodiments, a filtering workflow comprises steps which discard false transcripts, while retaining those associated with actual clonotypes, thereby providing improved and increased cell recovery.

### SUMMARY

In accordance with various embodiments, a first computer-implemented method for identifying one or more V(D)J cells by filtering at least one data set comprising a plurality of sequence barcode records from a plurality of single cells is provided. The first method can comprise receiving, at a computing device, at least one data set comprising a plurality of sequence barcode records from a plurality of single cells, wherein each sequence barcode record in the at least one data set comprises at least one V(D)J sequence. One or more filters are selected with the computing device from a plurality of filters based on whether the at least one data set includes gene expression data. If gene expression data is absent from the at least one data set, then a first set of filters is selected, wherein the first set of filters includes a second set of filters configured to remove a first sequence barcode record from the at least one data set including the first sequence barcode record and a second sequence barcode record where the first and second sequence barcode records comprise an identical V(D)J sequence. If gene expression data is present in the at least one data set, then a third set of filters is selected, wherein the third set of filters includes the first set of filters minus the second set of filters. The method further includes generating, with the computing device, a filtered data set that identifies one or more V(D)J cells from the plurality of single cells by filtering the at least one data set using the selected filters.

In accordance with various embodiments, the first set of filters includes initial filters configured to remove sequence barcode records from the first data set that have a transcript expression level below a threshold.

In accordance with various embodiments, the initial filters include a first filter configured to remove a sequence barcode record (e.g., barcode) that has less than a predetermined threshold of surviving non-solo unique molecular identifiers (UMIs).

In accordance with various embodiments, the initial filters include a second filter configured to remove a sequence barcode record that has less than a predetermined threshold of surviving non-solo UMIs and has less than a predetermined threshold of UMIs.

In accordance with various embodiments, the initial filters include a third filter configured to remove a sequence barcode record that does not include a contig having a V annotation.

In accordance with various embodiments, the initial filters include a fourth filter configured to remove a sequence barcode record that includes exactly one productive contig and includes a junction support contig.

In accordance with various embodiments, the initial filters include a fifth filter configured to remove a sequence barcode record that does not include a confident contig.

In accordance with various embodiments, the initial filters include a sixth filter configured to remove a sequence barcode record that has a maximum number of reads among the surviving UMIs that is less than 3% of the N50 reads per UMI computed for each barcode.

In accordance with various embodiments, the initial filters include a seventh filter configured to remove a sequence barcode record having a contig that is included in a set of contigs sharing the same junction segment when certain criteria is satisfied as detailed below.

In accordance with various embodiments, the initial filters include an eighth filter configured to remove a first sequence barcode record of a pair of sequence barcode records that share the same junction when the first sequence barcode record satisfies certain criteria as detailed below.

In accordance with various embodiments, the first set of filters includes chain sharing-based filters configured to remove a clone that shares a chain with another clone.

In accordance with various embodiments, the chain sharing-based filters include a ninth filter configured to remove a sequence barcode record based on V segments that appear in a set of contigs sharing the same CDR3 nucleotide.

In accordance with various embodiments, the chain sharing-based filters include a tenth filter configured to remove a sequence barcode record based on a set of junctions associated with two or more high confidence contigs.

In accordance with various embodiments, the chain sharing-based filters include an eleventh filter configured the same as the tenth filter except that the eleventh filters is only applicable to sequence barcode records with exactly two high confidence contigs and having only a single UMI supporting the junction of one of the two contigs.

In accordance with various embodiments, the first set of filters includes single cell filters configured to remove barcodes that contain material from two or more cells, cell fragments, or individual mRNA molecules so that barcodes that contain material from single cells remains.

In accordance with various embodiments, the single cell filters include a twelfth filter configured to remove sequence barcode records not called cells in a pipeline for processing and transforming sequencing data into the at least one data set.

In accordance with various embodiments, the single cell filters include a thirteenth filter configured to remove sequence barcode records having more than four productive contigs.

In accordance with various embodiments, the single cell filters include a fourteenth filter configured to remove some exact subclonotypes that appear to be background mRNA or a phenotypically similar phenomenon by virtue of their relationship to other exact subclonotypes.

In accordance with various embodiments, the single cell filters include a fifteenth filter configured to remove spurious exact subclonotypes using cross-library information.

In accordance with various embodiments, the single cell filters include a sixteenth filter configured to remove duplicated sequence barcode records within an exact subclonotype.

In accordance with various embodiments, the single cell filters include a seventeenth filter configured to remove rare artifacts arising from partition contamination, e.g. gel bead contamination.

In accordance with various embodiments, the single cell filters include an eighteenth filter configured to remove some four-chain clonotypes that might represent doublets.

In accordance with various embodiments, the single cell filters include a nineteenth filter configured to remove exact subclonotypes having multiple chains that are all of the same type.

In accordance with various embodiments, the single cell filters include a twentieth filter configured to disintegrate some single-chain clonotypes into single cells.

In accordance with various embodiments, the single cell filters include a twenty-first filter configured to remove some B cells having UMI counts below a threshold.

In accordance with various embodiments, the single cell filters include a twenty-second filter configured to remove some B cells having UMI counts below a threshold relative to UMI counts in a given clonotype.

In accordance with various embodiments, the single cell filters include a twenty-third filter configured to remove cells called by the V(D)J pipeline of the Cell Ranger™ Software but not by the gene expression (GEX) pipeline of the Cell Ranger™ Software.

In accordance with various embodiments, the single cell filters include a twenty-fourth filter configured to remove some sequence barcode records that appear to represent doublets.

In accordance with various embodiments, the single cell filters include a twenty-fifth filter configured to remove some sequence barcode records that appear to be contaminants based on the chain signature of the sequence barcode records.

In accordance with various embodiments, the single cell filters include a twenty-sixth filter configured to prevent the merger of some single-chain clonotypes into other clonotypes.

In accordance with various embodiments, the single cell filters include a twenty-seventh filter configured to remove cells having a chain that is likely spurious.

In accordance with various embodiments, the single cell filters include a twenty-eighth filter configured to remove exact subclonotypes having a position with low quality scores.

In accordance with various embodiments, the second set of filters are gene expression (GEX) data dependent and include the seventh, eighth, tenth, eleventh, fourteenth, twenty-first, and twenty-second filters.

In accordance with various embodiments, the at least one data set comprises a first data set and a second data set, and wherein the sequence barcode records of the second data set comprise a plurality of gene expression nucleic acid sequence barcode records such that gene expression data is present in the at least one data set.

In accordance with various embodiments, each gene expression nucleic acid sequence barcode record comprises at least one cDNA sequence.

In accordance with various embodiments, the sequence barcode records of the first data set are from the same plurality of single cells as the sequence barcode records of the second data set.

In accordance with various embodiments, the first method further comprises generating, with the computing device, a modified second data set by determining sequence barcode records of the plurality of gene expression nucleic acid sequence barcode records in the second data set that are associated with a single cell of the plurality of single cells.

In accordance with various embodiments, the plurality of gene expression nucleic acid sequence barcode records include data associated with a plurality of partitions wherein each partition comprises a single cell and data associated with partitions comprising two or more cells, and wherein generating the modified second data set comprises removing from the second data set the data associated with partitions comprising two or more cells.

In accordance with various embodiments, the third set of filters includes a first subset of filters and a second subset of filters, and wherein generating the filtered data set comprises: generating, with the computing device, a third data set by filtering the sequence barcode records of the first data set using the first subset of filters; generating, with the computing device, a fourth data set by removing from the third data set any sequence barcode record in the third data set that is absent from the modified second data set; and generating, with the computing device, the filtered data set by filtering the fourth data set using the second subset of filters.

In accordance with various embodiments, the first method further comprises generating, with the computing device, a modified filtered data set by grouping the one or more V(D)J cells in the filtered data set into at least one clonotype.

In accordance with various embodiments, the first method further comprises: identifying, with the computing device and from the modified filtered data set, a clonotype having a quantity of chains greater than a predetermined threshold, the clonotype including a plurality of subclonotypes; and replacing, with the computing device, the identified clonotype with each of the plurality of subclonotypes such that each subclonotype becomes a respective clonotype.

In accordance with various embodiments, each of the one or more V(D)J cells are a T cell and/or a B cell.

In accordance with various embodiments, the first method further comprises detecting, with the computing device, whether the at least one data set includes gene expression data.

In accordance with various embodiments, a first system for identifying one or more V(D)J cells by filtering at least one data set comprising a plurality of sequence barcode records from a plurality of single cells is provided. The first system can comprise a memory and a processor in communication with the memory. The processor is configured to perform the operations comprising the steps of the various embodiments of the first method.

In accordance with various embodiments, a first non-transitory computer-readable medium storing instructions, which when executed by a processor, cause the processor to perform operations for identifying one or more V(D)J cells by filtering at least one data set comprising a plurality of sequence barcode records from a plurality of single cells is provided. The operations can comprise the steps of the various embodiments of the first method.

In accordance with various embodiments, a second computer-implemented method for identifying one or more V(D)J cells by filtering at least one data set comprising a plurality of sequence barcode records from a plurality of single cells is provided. The second method includes receiving, at a computing device, a first data set and a second data set, each comprising a plurality of sequence barcode records from a plurality of single cells, wherein each sequence barcode record in the first and second data sets comprises at least one V(D)J sequence, and wherein the sequence barcode records of the second data set comprise a plurality of gene expression nucleic acid sequence barcode records that are associated with a single cell of the plurality of single cells. A third data set is generated with the computing device by filtering the first data set using a first set of filters configured to remove sequence barcode records from the first data set that have a transcript expression level below a threshold. A fourth data set is generated with the computing device by removing from the third data set any sequence barcode record in the third data set that is absent from the second data set. A fifth data set is generated with the computing device that identifies one or more V(D)J cells from the plurality of single cells by filtering the fourth data set using a second set of filters configured to remove from the second filtered data set sequence barcode records that contain material from two or more cells, cell fragments, or individual mRNA molecules.

In accordance with various embodiments, the second set of filters include the single cell filters described above except for the GEX data dependent filters described above.

In accordance with various embodiments, the sequence barcode records of the first data set are from the same plurality of single cells as the sequence barcode records of the second data set.

In accordance with various embodiments, the second method further comprises generating a modified fifth data set by grouping the one or more V(D)J cells in the fifth data set into at least one clonotype.

In accordance with various embodiments, the second method further comprises identifying, from the modified fifth data set, a clonotype having a quantity of chains greater than a predetermined threshold, the clonotype including a plurality of subclonotypes; and replacing the identified clonotype with each of the plurality of subclonotypes such that each subclonotype becomes a respective clonotype.

In accordance with various embodiments, a second system for identifying one or more V(D)J cells by filtering at least one data set comprising a plurality of sequence barcode records from a plurality of single cells is provided. The second system can comprise a memory and a processor in communication with the memory. The processor is configured to perform the operations comprising the steps of the various embodiments of the second method.

In accordance with various embodiments, a second non-transitory computer-readable medium storing instructions, which when executed by a processor, cause the processor to perform operations for identifying one or more V(D)J cells by filtering at least one data set comprising a plurality of sequence barcode records from a plurality of single cells is provided. The operations can comprise the steps of the various embodiments of the second method.

In accordance with various embodiments, a third computer-implemented method for identifying one or more V(D)J cells by filtering at least one data set comprising a plurality of sequence barcode records from a plurality of single cells is provided. The third method includes receiving, at a computing device, a data set comprising a plurality of sequence barcode records from a plurality of single cells, wherein each sequence barcode record in the data set comprises at least one V(D)J sequence; generating, with the computing device, a first filtered data set by filtering the data set using a first set of filters configured to remove sequence barcode records from the data set that have a transcript expression level below a threshold; generating, with the computing device, a second filtered data set by filtering the first filtered data set using a second set of filters configured to remove from the first filtered data set a clone that shares a chain with another clone; and generating, with the computing device, a third filtered data set that identifies one or more V(D)J cells from the plurality of single cells by filtering the second filtered data set using a third set of filters configured to remove from the second filtered data set sequence barcode records that contain material from two or more cells, cell fragments, or individual mRNA molecules.

In accordance with various embodiments, the third method further comprises generating a modified fifth data set by grouping the one or more V(D)J cells in the fifth data set into at least one clonotype.

In accordance with various embodiments, a third system for identifying one or more V(D)J cells by filtering at least one data set comprising a plurality of sequence barcode records from a plurality of single cells is provided. The third system can comprise a memory and a processor in communication with the memory. The processor is configured to perform the operations comprising the steps of the various embodiments of the third method.

In accordance with various embodiments, a third non-transitory computer-readable medium storing instructions, which when executed by a processor, cause the processor to perform operations for identifying one or more V(D)J cells by filtering at least one data set comprising a plurality of sequence barcode records from a plurality of single cells is provided. The operations can comprise the steps of the various embodiments of the third method.

In accordance with various embodiments, a fourth computer-implemented method for identifying one or more V(D)J cells by filtering at least one data set comprising a plurality of sequence barcode records from a plurality of single cells is provided. The fourth method includes: receiving, at a computing device, a first data set and optionally a GEX cells data set, each comprising a plurality of sequence barcode records from a plurality of single cells, wherein each sequence barcode record in the first set comprises at least one V(D)J sequence, and wherein the sequence barcode records of the GEX cells data set comprise a plurality of gene expression nucleic acid sequence barcode records that are associated with or identified as a single cell among the plurality of single cells; optionally generating, with the computing device, a second data set by filtering the first data set using a first set of filters, in non-limiting embodiments configured to remove sequence barcode records from the first data set that have a transcript expression level below a threshold; receiving, at a computing device, GEX cells data set, comprising a plurality of sequence barcode records from a plurality of single cells, wherein the sequence barcode records of the GEX cells data set comprise a plurality of gene expression nucleic acid sequence barcode records that are associated with a single cell of the plurality of single cells; generating, with the computing device, a third data set by removing from the second data set any sequence barcode record that is absent from the GEX cells data set; wherein in non-limiting embodiments generating the third data set comprises comparing the barcode records in the second data set to the barcode records in the GEX cells data set and removing from the second data set any sequence barcode record that is absent from the GEX cells data set to generate a third data set; and generating, with a computing device, a fourth data set (with GEX) that comprise one or more V(D)J cells from the plurality of single cells by filtering the third data set using a fourth set of filter(s), in non-limiting embodiments configured to remove from the third data set sequence barcode records that contain material from two or more cells, cell fragments, or individual mRNA molecules, for example mRNA molecules which are not associated with a particular cell.

In accordance with various embodiments, the first set of filters of the fourth method are the initial filters described above.

In accordance with various embodiments, the fourth set of filters of the fourth method are the single cell filters described above excluding the GEX data dependent filters. In non-limiting embodiments, the fourth set of filters include the twelfth filter, thirteenth filter, fifteenth through the twentieth filters, and twenty-third through the twenty-eighth filters described above.

In accordance with various embodiments, the plurality of sequence barcode records from the plurality of single cells are generated by sequencing a plurality of single cells. In non-limiting embodiments, the plurality of sequence barcode records from the plurality of single cells are generated from cells in partitions. Any suitable partitioning method that allows for analysis of nucleic acids from single cells is contemplated. In non-limiting embodiments, the partitions comprise solid support. In non-limiting embodiments, the partitions are wells. In non-limiting embodiments, each cell and/or nucleus could serve as a partition. In non-limiting embodiments, the partitions are droplets, including droplets in emulsion, wherein the droplets in emulsion are generated by any suitable method. In non-limiting embodiments, the droplets are formed by a microfluidic device. In non-limiting embodiments, the partition is a GEM. In non-limiting embodiments, the plurality of sequence barcode records from the plurality of single cells are generated by any suitable single cell sequencing method.

In accordance with various embodiments, at least one of the plurality of gene expression nucleic acid sequence barcode records in the GEX cells data set comprises at least one cDNA sequence. The In non-limiting embodiments, the at least one cDNA is full length cDNA.

In accordance with various embodiments, the sequence barcode records of the first data set are from the same plurality of single cells as the sequence barcode records of the GEX cells data set.

In accordance with various embodiments, the fourth method further comprises generating a fifth data set by grouping the one or more V(D)J cells in the fourth data set into at least one clonotype.

In accordance with various embodiments, the fourth method further comprises identifying, from the fifth data set, at least one clonotype having a number/quantity of chains greater than a predetermined threshold, the clonotype including a plurality of subclonotypes; and replacing the identified clonotype with each of the plurality of subclonotypes such that each subclonotype becomes a respective clonotype.

In accordance with various embodiments, a fourth system for identifying one or more V(D)J cells by filtering at least one data set comprising a plurality of sequence barcode records from a plurality of single cells is provided. The fourth system can comprise a memory and a processor in communication with the memory. The processor is configured to perform the operations comprising the steps of the various embodiments of the fourth method.

In accordance with various embodiments, a fourth non-transitory computer-readable medium storing instructions, which when executed by a processor, cause the processor to perform operations for identifying one or more V(D)J cells by filtering at least one data set comprising a plurality of sequence barcode records from a plurality of single cells is provided. The operations can comprise the steps of the various embodiments of the fourth method.

These and other aspects and implementations are discussed in detail herein. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIG. 1 is an example system for identifying one or more V(D)J cells in accordance with various embodiments.

FIG. 2 is an example workflow diagram for identifying one or more V(D)J cells in the absence of gene expression data, in accordance with various embodiments.

FIG. 3 is a block diagram of an example method for identifying one or more V(D)J cells in the presence of gene expression data, in accordance with various embodiments.

FIG. 4 is an example workflow diagram for identifying one or more V(D)J cells in the presence of gene expression data, in accordance with various embodiments.

FIGS. 5A and 5B are graphs comparing V(D)J cell recovery between certain filters being active versus inactive, in accordance with various embodiments.

FIG. 6 is a block diagram that illustrates a computer system, upon which embodiments, or portions of the embodiments, may be implemented, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

Unless defined otherwise, all technical and scientific terms used herein have the same meaning as commonly understood by one of ordinary skill in the art to which the various embodiments belong.

All publications mentioned herein are incorporated herein by reference for the purpose of describing and disclosing devices, compositions, formulations and methodologies which are described in the publication and which might be used in connection with the present disclosure.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina, the GRIDION and PROMETHION Systems of Oxford Nanopore Technologies, PACBIO SEQUEL Systems of Pacific Biosciences, and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.) which denotes a single or a grouping of genes (in DNA or RNA) that have undergone changes as referenced against a particular species or sub-populations within a particular species due to mutations, recombination/crossover or genetic drift.

The term “B cells”, also known as B lymphocytes, refer to a type of white blood cell of the small lymphocyte subtype. They function in the humoral immunity component of the adaptive immune system by secreting antibodies. Additionally, B cells present antigen (they are also classified as professional antigen-presenting cells (APCs)) and secrete cytokines. In mammals, B cells mature in the bone marrow, which is at the core of most bones. In birds, B cells mature in the bursa of Fabricius, a lymphoid organ where they were first discovered by Chang and Glick, (B for bursa) and not from bone marrow as commonly believed. B cells, unlike the other two classes of lymphocytes, T cells and natural killer cells, express B cell receptors (BCRs) on their cell membrane. BCRs allow the B cell to bind to a specific antigen, against which it will initiate an antibody response.

The term “T cell”, also known as T lymphocytes, refer to a type of an adaptive immune cell. T cells develops in the thymus gland, hence the name T cell, and play a central role in the immune response of the body. T cells can be distinguished from other lymphocytes by the presence of a T cell receptor (TCR) on the cell surface. These immune cells originate as precursor cells, derived from bone marrow, and then develop into several distinct types of T cells once they have migrated to the thymus gland. T cell differentiation continues even after they have left the thymus. T cells include, but are not limited to, helper T cells, cytotoxic T cells, memory T cells, regulatory T cells, and killer T cells. Helper T cells stimulate B cells to make antibodies and help killer cells develop. Based on the T cell receptor chain, T cells can also include T cells that express αβ TCR chains, T cells that express γδ TCR chains, as well as unique TCR co-expressors (i.e., hybrid αβ-γδ T cells) that co-express the αβ and γδ TCR chains.

T cells can also include engineered T cells that can attack specific cancer cells. A patient's T cells can be collected and genetically engineered to produce chimeric antigen receptors (CAR). These engineered T cells are called CAR T cells, which forms the basis of the developing technology called CAR-T therapy. These engineered CAR T cells are grown by the billions in the laboratory and then infused into a patient's body, where the cells are designed to multiply and recognize the cancer cells that express the specific protein. This technology, also called adoptive cell transfer is emerging as a potential next-generation immunotherapy treatment.

T cells, such as the killer T cells can directly kill cells that have already been infected by a foreign invader. T cells can also use cytokines as messenger molecules to send chemical instructions to the rest of the immune system to ramp up its response. Activating T cells against cancer cells is the basis behind checkpoint inhibitors, a relatively new class of immunotherapy drugs that have recently been approved to treat lung cancer, melanoma, and other difficult cancers. Cancer cells often evade patrolling T cells by sending signals that make them seem harmless. Checkpoint inhibitors disrupt those signals and prompt the T cells to attack the cancer cells.

The term “naïve”, as used herein, can refer to B-lymphocytes or T-lymphocytes that have not yet reacted with an epitope of an antigen or that have a cellular phenotype consistent with that of a lymphocyte that has not yet responded to antigen-specific activation after clonal licensing.

The term “Fab”, also referred to as an antigen-binding fragment, refers to the variable portions of an antibody molecule with a paratope that enables the binding of a given epitope of a cognate antigen. The amino acid and nucleotide sequences of the Fab portion of antibody molecules are hypervariable. This is in contrast to the “Fc” or crystallizable fragment, which is relatively constant and encodes the isotype for a given antibody; this region can also confer additional functional capacity through processes such as antibody-dependent complement deposition, cellular cytotoxicity, cellular trogocytosis, and cellular phagocytosis.

The phrase “clonal selection” refers to the selection and activation of specific B lymphocytes and T lymphocytes by the binding of epitopes to B cell receptors or T cell receptors with a corresponding fit and the subsequent elimination (negative selection) or licensing for clonal expansion (positive selection) of a B or T lymphocyte after binding of an antigenic determinant.

The phrase “clonal expansion” refers to the proliferation of B lymphocytes and T lymphocytes activated by clonal selection in order to produce a clonal population of daughter cells with the same antigen specificity and functional capacity. In the case of T lymphocytes this antigen specificity is exact at the nucleotide and protein level and in the case of B lymphocytes this antigen specificity can be exact at the nucleotide and protein level or mutated relative to the parent population by mutations at the nucleotide level (and by extension the protein level). This enables the body to have sufficient numbers of antigen-specific lymphocytes to mount an effective immune response.

The term “cytokines” refers to a wide variety of intercellular regulatory proteins produced by many different cells in the body which ultimately control every aspect of body defense. Cytokines activate and deactivate phagocytes and immune defense cells, increase or decrease the functions of the different immune defense cells, and promote or inhibit a variety of nonspecific body defenses.

The phrase “T4-helper lymphocytes”, also referred to as helper cells, refer to a type of white blood cell that orchestrate the immune response and enhance the activities of the killer T-cells (those that destroy pathogens) and B cells (antibody and immunoglobulin producers).

The phrase “affinity maturation” refers to the gradual modification of the paratope and entire B cell receptor as a result of somatic hypermutation. B lymphocytes with higher affinity B cell receptors that can 1) bind the epitope more tightly and 2) therefore bind the epitope for a longer period of time are able to proliferate more and survive longer. These B cells can eventually differentiate into plasma cells, which secrete their antibodies and form the basis of serum-mediated immunity.

The phrase “somatic hypermutation” (SHM) refers to a cellular mechanism by which the adaptive immune system adapts to foreign elements confronting it (e.g. viruses, bacteria, biomolecules). A major component of the process of affinity maturation, SHM diversifies B cell receptors used to recognize foreign elements (antigens) and allows the immune system to adapt its response to new threats during the lifetime of an organism. Somatic hypermutation involves a programmed process of mutation predominantly affecting select framework and complementarity-determining regions of immunoglobulin genes. Unlike germline mutation, SHM operates at the level of an organism's individual immune cells. These mutations are not transmitted to the organism's offspring, but are transmitted to daughter cells of individual B cell clones. Mistargeted somatic hypermutation is a likely mechanism in the development of B cell lymphomas and many other cancers. Somatic hypermutation can also lead to the acquisition of non-V(D)J template DNA within B cell receptor sequences, such as LAIR1 insertions in malaria-specific neutralizing antibodies.

Somatic hypermutation is a distinct diversification mechanism from isotype switching (also called class switching). Mutations acquired during somatic hypermutation eventually lead to isotype switching, in which a B cell's antibody can be coupled to different functions by switching to a different Fc/constant region sequence. Isotype switching is an irreversible process, in that once a B cell has switched from a given constant region (e.g. IGHM) to a new constant region (e.g. IGHA1) it can no longer use the IgM constant region as the DNA encoding the IgM Fc is excised and removed during isotype switching.

The term “contig”, originating from the term “contiguous”, refers to a set of overlapping DNA segments that together represent a consensus region of DNA. In bottom-up sequencing projects, a contig refers to overlapping sequence data (reads); in top-down sequencing projects, contig refers to the overlapping clones that form a physical map of the genome that is used to guide sequencing and assembly. Contigs can thus refer both to overlapping DNA sequences and to overlapping physical segments (fragments) contained in clones depending on the context. Note that clone, in reference to overlapping clones, refers to individual bacteria or constructs (e.g. phagemids, cosmids, etc.) containing distinct insertions of genomes that were utilized in early efforts to map genomes.

The phrase “heavy chain” refers to the large polypeptide subunit of an antibody (immunoglobulin). The first recombination event to occur is between one D and one J gene segment of the heavy chain locus. Any chromosomal DNA between these two gene segments is deleted. This D-J recombination is followed by the joining of one V gene segment, from a region upstream of the newly formed DJ complex, forming a rearranged V(D)J gene segment. All other gene segments between V and D segments are now deleted from the cell's genome. Primary transcript (unspliced RNA) is generated containing the V(D)J region of the heavy chain and both the constant mu and delta chains (Cμ and Cδ) (i.e., the primary transcript contains the segments: V-D-J-Cμ-Cδ). The primary RNA is processed to add a polyadenylated (poly-A) tail after the Cμ chain and to remove sequence between the V(D)J segment and this constant gene segment. Translation of this mRNA leads to the production of the IgM heavy chain protein and the IgD heavy chain protein (its splice variant). Expression of the immunoglobulin heavy chain with one or more surrogate light chains constitutes the pre-B cell receptor that allows a B cell to undergo selection and maturation.

The phrase “light chain” refers to the small polypeptide subunit of an antibody (immunoglobulin). The kappa (κ) and lambda (λ) chains of the immunoglobulin light chain loci rearrange in a very similar way, except that the light chains lack a D segment. In other words, the first step of recombination for the light chains involves the joining of the V and J chains to give a VJ complex before the addition of the constant chain gene during primary transcription. Translation of the spliced mRNA for either the kappa or lambda chains results in formation of the Ig κ or Ig λ light chain protein. Assembly of the Ig μ heavy chain and one of the light chains results in the formation of membrane bound form of the immunoglobulin IgM that is expressed on the surface of the immature B cell. B cells may express up to two heavy chains and/or two light chains in respectively rare and uncommon instances through a phenomenon known as allelic inclusion. This phenomenon can only be directly observed using single-cell technologies, though it can be inferred with a degree of uncertainty using a combination of bulk sequencing technologies and probabilistic inference via an extension of the birthday paradox.

The phrase “complementarity-determining regions” (CDRs) refers to part of the variable chains in immunoglobulins (antibodies) and T cell receptors, generated by B cells and T cells respectively, where these molecules are particularly hypervariable. The antigen-binding site of most antibodies and T cell receptors is typically distributed across these CDRs, collectively forming a paratope. However, there are many documented examples of paratopes that enable antigen recognition that fall outside of the CDRs. As the most variable parts of the molecules, CDRs are crucial to the diversity of antigen specificities and immune cell receptor sequences generated by lymphocytes.

In some aspects, the methods and systems described herein can provide for the determination of the sequence of long individual nucleic acid molecules and/or the identification of direct molecular linkage as between two sequence segments separated by long stretches of sequence, which permit the identification and use of long range sequence information, wherein such sequencing information is obtained using methods that have the advantages of the extremely low sequencing error rates and high throughput of short read sequencing technologies. The methods and systems described herein can segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long range molecular context can be preserved through a sequencing process, and can be preserved through the targeted enrichment process used in targeted sequencing approaches described herein, where no other sequencing approach has shown this ability.

In some aspects, sequence information from smaller fragments may retain the original long range molecular sequence context through the use of a tagging procedure, including the addition of barcodes as described herein or known in the art. In specific examples, fragments originating from the same original longer individual nucleic acid molecule can be tagged with a common barcode, such that any later sequence reads from those fragments can be attributed to that originating longer individual nucleic acid molecule. Such barcodes can be added using any method known in the art, including addition of barcode sequences during amplification methods that amplify segments of the individual nucleic acid molecules as well as insertion of barcodes into the original individual nucleic acid molecules using transposons, including methods such as those described in Amini et al., Nature Genetics 46: 1343-1349 (2014) (advance online publication on Oct. 29, 2014), which is hereby incorporated by reference in its entirety for all purposes and in particular for all teachings related to adding adaptor and other oligonucleotides using transposons. Once nucleic acids have been tagged using such methods, the resultant tagged fragments can be enriched using methods described herein such that the population of fragments represents targeted regions of the genome. As such, sequence reads from that population allows for targeted sequencing of select regions of the genome, and those sequence reads can also be attributed to the originating nucleic acid molecules, thus preserving the original long range molecular sequence context. The sequence reads can be obtained using any sequencing methods and platforms known in the art and described herein. In some aspects, such methods and systems are useful for assembly of complete V(D)J sequences.

Methods of processing and sequencing nucleic acids in accordance with the methods and systems described in the present application are also described in further detail in U.S. Ser. Nos. 14/316,383; WO2015200893, WO2018119447 and WO2018075693 which are herein incorporated by reference in their entirety for all purposes and in particular for all written description, figures and working examples directed to processing nucleic acids and sequencing and other characterizations of genomic material.

In general, the methods and systems described herein accomplish sequencing of nucleic acid molecules including, but not limited to, DNA (e.g., genomic DNA), RNA (e.g., mRNA, including full-length mRNA transcripts, and small RNAs, such as miRNA, tRNA, and rRNA), and cDNA. In various embodiments, the methods and systems described herein accomplish genomic sequencing of nucleic acid molecules (e.g., DNA, RNA, and mRNA). In various embodiments, the methods and systems described herein accomplish genomic sequencing of immune cell receptor sequences (e.g., DNA, RNA, and mRNA). In various embodiments, the methods and systems described herein can accomplish transcriptome sequencing, e.g., whole transcriptome sequencing of mRNA encoding immune cell receptors. In some embodiments, the methods and systems described herein can also accomplish targeted genomic sequencing of nucleic acid molecules (e.g., DNA, RNA, and mRNA). In various embodiments, the methods and systems described herein accomplish single cell genomic sequencing, for example, single cell genomic sequencing of nucleic acid molecules (e.g., RNA and mRNA) encoding immune cell receptors of single cells, such as B cell receptors (BCRs) and T cell receptors (TCRs).

In various embodiments, the methods and systems described herein can include high-throughput sequencing technologies, e.g., high-throughput DNA and RNA sequencing technologies, and/or any data set generated from such high-throughput sequencing technologies. In various embodiments, the methods and systems described herein can include high-throughput, higher accuracy short-read DNA and RNA sequencing technologies. In various embodiments, the methods and systems described herein can include long-read RNA sequencing, e.g., by sequencing cDNA transcripts in their entirety without assembly. In various embodiments, the methods and systems described herein can also, for example, segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long-range molecular context is not only preserved through a sequencing process, but is also preserved through the targeted enrichment process used in targeted sequencing approaches. In various embodiments, the methods and systems described herein can include gene expression data, including one or more data sets generated from sequencing an entire genome, or a portion of a genome, such as a gene, a segmented long nucleic acid molecule, and/or a combination of genes, gene fragments, and/or segmented long nucleic acid molecules. In various embodiments, the methods and systems described herein can include whole transcriptome data.

In general, the methods and systems described herein are directed to single cell analysis (including single- and multi-modal analyses) of genomic sequencing of nucleic acids (e.g., RNA and mRNA) encoding immune cell receptors of single cells, such as B cell receptors (BCRs) and T cell receptors (TCRs). Single cell analysis, including single cell multi-modal analyses (e.g., single cell immune cell receptor sequencing combined with, for example, gene expression, protein expression, and/or antigen capture technologies), as well as processing and sequencing of nucleic acids, in accordance with the methods and systems described in the present application are described in further detail, for example, in U.S. Pat. Nos. 9,689,024; 9,701,998; 10,011,872; 10,221,442; 10,337,061; 10,550,429; 10,273,541; and U.S. Pat. Pub. 20180105808, which are all herein incorporated by reference in their entirety for all purposes and in particular for all written description, figures and working examples directed to processing nucleic acids and sequencing and other characterizations of genomic material.

V(D)J recombination is a genetic recombination mechanism that occurs in developing lymphocytes during the early stages of T and B cell maturation. Through somatic recombination, this mechanism produces a highly diverse repertoire of antibodies/immunoglobulins and T cell receptors (TCRs) found in B cells and T cells, respectively. This process is a defining feature of the adaptive immune system and these receptors are defining features of adaptive immune cells.

V(DD)J recombination is a genetic recombination mechanism that, while discovered decades earlier, was not truly understood until recently given its non-adherence to classical rules to V(D)J recombination. However, understanding this mechanism have been a clear need, since tandem fusions of D-D genes can result in long CDR3s (24+ amino acids) or ultralong CDR3s (28+ amino acids). Though relatively rare, these long CDR3s are very biologically relevant, as they can be found in broadly neutralizing antibodies. See, for reference, https://www.ncbi.nlm.nih.gov/pmc/articles/PMC7605257/.

V(D)J recombination occurs in the primary lymphoid organs (bone marrow for B cells and thymus for T cells) and in a generally random fashion. The process leads to the rearranging of variable (V), joining (J), and in some cases, diversity (D) gene segments. As discussed above, the heavy chain possesses numerous V, D, and J gene segments, while the light chain possesses only V and J gene segments. The process ultimately results in novel amino acid sequences in the antigen-binding regions of immunoglobulins and TCRs that allow for the recognition of antigens from nearly all pathogens including, for example, bacteria, viruses, and parasites. Furthermore, the recognition can also be allergic in nature or may match host tissues and lead to autoimmunity.

Human antibody molecules, including B cell receptors (BCRs), include both heavy and light chains, each of which contains both constant (C) and variable (V) regions, and are genetically encoded on three loci. The first is the immunoglobulin heavy locus on chromosome 14, containing the gene segments for the immunoglobulin heavy chain. The second is the immunoglobulin kappa (κ) locus on chromosome 2, containing the gene segments for part of the immunoglobulin light chain. The third is the immunoglobulin lambda (λ) locus on chromosome 22, containing the gene segments for the remainder of the immunoglobulin light chain.

Each heavy or light chain contains multiple copies of different types of gene segments for the variable regions of the antibody proteins. For example, the human immunoglobulin heavy chain region contains two C gene segments (Cμ and Cδ), 44 V gene segments, 27 D gene segments and 6 J gene segments. The number of given segments present in any individual can vary, as these gene segments are carried in haplotypes; for this reason, inference of both the alleles present within an individuals and the germline sequence of those alleles is an important step in correctly identifying B cell clonotypes. The light chains possess two C gene segments (Cλ and Cκ) and numerous V and J gene segments, but do not have D gene segments. DNA rearrangement causes one copy of each type of gene segment to mate with any given lymphocyte, generating a substantial antibody repertoire. Approximately 10¹⁴ combinations are possible, with 1.5×10² to 3×10³ potentially removed via self-reactivity.

Accordingly, each naïve B cell makes an antibody with a unique Fab site through a series of gene recombinations, and later mutations, with the specific molecules of the given antibody attaching to the B cell's surface as a B cell receptor (BCR). These BCRs are then available to react with epitopes of an antigen.

When the immune system encounters an antigen, epitopes of that antigen will be presented to many B lymphocytes. B lymphocytes must first rearrange a heavy chain that enables pre-B cell receptor ligand binding. B lymphocytes that bind multivalent self-targets after rearrangement of the light chain too strongly are eliminated and die or undergo a secondary recombination event, while B cells that do not bind self-targets too strongly are licensed to exit the bone marrow. The latter becomes available to respond to non-self antigens and to undergo clonal expansion. This process is known as clonal selection.

Cytokines produced by activated T4-helper lymphocytes enable those activated B-lymphocytes (B cells) to rapidly proliferate to produce large clones of thousands of identical B cells. More specifically, when under threat (i.e., via bacteria, virus, etc.), the body releases white blood cells by the immune system. The T4 lymphocytes help the response to a threat by triggering the maturation of other types of white blood cell. They produce special proteins, called cytokines, have plural functions, including the ability to summon all of the other immune cells to the area, and also the ability to cause nearby cells to differentiate (become specialized) into mature B cells and T-cells.

Accordingly, while only a few B cells in the body may have an antibody molecule that can bind a particular epitope, eventually many thousands of cells are produced with the right specificity, allowing the body's immune system to act en masse. This is referred to as clonal expansion. Natural phenomena such as IgA deficiency and murine transgenic models have shown that there are multiple paths by which a B cell receptor can acquire novel antigen specificity even from a very limited repertoire through the processes of somatic hypermutation and affinity maturation.

As the B cells proliferate, they undergo affinity maturation as a result of somatic hypermutation. This allows the B cells to “fine-tune” the paratopes of the antibody to more effectively fit with the recognized epitopes. B cells with high affinity B cell receptors on their surface bind epitopes more tightly and for a longer period of time, which enables these cells to selectively proliferate. Over the course of this proliferation and expansion, these variant B cells differentiate into plasma cells that synthesize and secrete vast quantities of antibodies with Fab sites that fit the target epitopes very precisely.

The phrase “immune cell” refers to a cell that is part of the immune system and that helps the body fight infections and other diseases. Immune cells include innate immune cells (such as basophils, dendritic cells, neutrophils, etc.) that are the first line of body's defense and are deployed to help attack the invading foreign cells (e.g., cancer cells) and pathogens. The innate immune cells can quickly respond to foreign cells and pathogens to fight infection, battle a virus, or defend the body against bacteria. Immune cells can also include adaptive immune cells (such as lymphocytes including B cells and T cells). The adaptive immune cells can come into action when an invading foreign cells or pathogens slip through the first line of body's defense mechanism. The adaptive immune cells can take longer to develop, because their behaviors evolve from learned experiences, but they can tend to live longer than innate immune cells. Adaptive immune cells remember foreign invaders after their first encounter and fight them off the next time they enter the body. Both types of immune cells employ important natural defenses in helping the body fight foreign cells and pathogens for fighting infections and other diseases.

Accordingly, the immune cells of the disclosure can include, but are not limited to, neutrophils, eosinophils, basophils, mast cells, monocytes, macrophages, dendritic cells, natural killer cells, and lymphocytes (such as B cells and T cells). The immune cells of the disclosure can further include dual expresser cells or DE (such as unique dual-receptor-expressing lymphocytes that co-express functional B cell receptor (BCR) and T cell receptor (TCR)), cells with adaptive immune receptors that may diversify or may not diversify (including immune cells expressing a chimeric antigen receptor with a fixed nucleotide sequence or with the capacity to mutate), and TCR co-expressors (i.e., hybrid αβ-γδ T cells) that co-express both αβ and γδ TCR chains.

The phrase “immune cell receptor”, “immune receptor”, or “immunologic receptor” refers to a receptor or immune cell receptor sequence, usually on a cell membrane, which can recognize components of pathogenic microorganisms (e.g., components of bacterial cell wall, bacterial flagella or viral nucleic acids) and foreign cells (e.g., cancer cells), which are foreign and not found naturally on the host cells, or binds to a target molecule (for example, a cytokine), and causes a response in the immune system. The immune cell receptors of the immune system can include, but are not limited to, pattern recognition receptors (PRRs), Toll-like receptors (TLRs), killer activated and killer inhibitor receptors (KARs and KIRs), complement receptors, Fc receptors, B cell receptors, and T cell receptors.

The phrase “immune cell receptor sequences” of an immune cell receptor include both heavy and light chains, each of which contains both constant (C) and variable (V) regions. For example, B cell receptors (BCRs) or B cell receptor sequences (including human antibody molecules) comprise of immunoglobulin heavy and light chains, each of which contains both constant (C) and variable (V) regions. Each heavy or light chain not only contains multiple copies of different types of gene segments for the variable regions of the antibody proteins, but also contains constant regions. For example, the BCR or human immunoglobulin heavy chain contains two (2) constant (Constant mu (Cμ) and delta (Cδ)) gene segments and forty four (44) Variable (V) gene segments, plus twenty seven (27) Diversity (D) gene segments, and six (6) Joining (J) gene segments. The BCR light chains also possess two (2) constant gene segments ((Constant lambda (Cλ) and kappa (Cκ)) and numerous V and J gene segments, but do not have any D gene segments. DNA rearrangement (i.e., recombination events) in developing B cells can cause one copy of each type of gene segment to go in any given lymphocyte, generating an enormous antibody repertoire. Accordingly, the primary transcript (unspliced RNA) of a BCR heavy chain can be generated containing the V(D)J region of the heavy chain and both the constant mu and delta chains (Cμ and Cδ), i.e., the heavy chain primary transcript can contains the segments: V-D-J-Cμ-Cδ). In case of the B cell receptor and human immunoglobulin light chain, the first step of recombination for the light chains involves the joining of the V and J chains to give a VJ complex before the addition of the constant chain gene during primary transcription. Translation of the spliced mRNA for either the constant κ (Cκ) or λ(Cλ) chains results in formation of the Ig κ or Igλ light chain protein.

In general, most T cell receptors (TCR) are composed of an alpha (a) chain and a beta (p) chain, each of which contains both constant (C) and variable (V) regions. Thus, the most common type of a T cell receptor is called an alpha-beta TCR because it is composed of two different chains, one α-chain and one beta β-chain. A less common type of TCR is the gamma-delta TCR, which contains a different set of chains, one gamma (γ) chain and one delta (δ) chain. The T cell receptor genes are similar to immunoglobulin genes for the BCR and undergo similar DNA rearrangement (i.e., recombination events) in developing T cells as for the B cells. For example, the alpha-beta TCR genes also contain multiple V, D, and J gene segments in their beta chains and V and J gene segments in their alpha chains, which are re-arranged during the development of the T cells to provide a cell with a unique T cell antigen receptor. Thus, the β-chain of the TCR can contain VO-DO-JO gene segments and constant domain (Cβ) genes resulting in a Vβ-Dβ-Jβ-Cβ sequence of the TCR β-chain. The re-arrangement of the alpha (α) chain of the TCR follows β chain rearrangement, and can include Vα-Jα gene segments and constant domain (Cα) genes resulting in a Vα-Jα-Cα sequence of the TCR α-chain. Similar to the alpha-beta TCRs, the TCR-γ chain is produced by V-J recombinations and can contain Vγ-Jγ gene segments and constant domain (CT) genes resulting in a Vγ-Jγ-Cγ sequence of the TCR γ-chain, while the TCR-δ chain is produced using V-D-J recombinations, and can contain Vδ-Dδ-Jδ gene segments and constant domain (Cδ) genes resulting in a Vδ-Dδ-Jδ-Cδ sequence of the TCR δ-chain.

The phrase “immune cell receptor constant region sequence” or “immune receptor constant region sequence” refers to the constant region or constant region sequence of an immune cell receptor. For example, the immune cell receptor constant region sequence or immune receptor constant region sequence can include, but is not limited to, the constant mu (Cμ) and delta (Cδ) region genes and sequences of a BCR and immunoglobulin heavy chain, the constant lambda (Cλ) and kappa (Cκ) region genes and sequences of a BCR and immunoglobulin light chain, the alpha constant (Cα) region genes and sequences of a TCR α-chain sequence, the beta constant (Cβ) region genes and sequences of a TCR β-chain sequence, the gamma constant (Cγ) region genes and sequences of a TCR γ-chain sequence, and the delta constant (Cδ) region genes and sequences of a TCR δ-chain sequence.

To understand what constitutes members of a clonotype, one can start with the original progenitor cell for a given lineage of B cells, this progenitor cell commonly referred to as the parent clone, which is a single cell to which all daughter cells will be genetically related, though their B cell receptors and exact antigen specificity may differ. Collectively, this parent clone and all its daughter cells constitute a clonotype. As stated above, accurate identification of the members of a clonotype is critical not just from a biological perspective, but also from the biomedical perspective, as correct identification of all of the members of a given clonotype can be useful in the identification and discovery of therapeutic antibodies, design of vaccines (e.g., what antibody lineages can be expanded by a vaccine or are expanded successfully or unsuccessfully by a vaccine), in the monitoring of B cell-mediated immune disease (e.g., myasthenia gravis and lupus), and in other settings. Known approaches that attempt to group immune cell receptor sequences into groups with shared antigen specificity or members of the same clonotype include 1) immcantation, 2) Clonify, 3) GLIPH, 4) TCRdist, 5) V(D)JTools, 6) MiXCR, 7) AbSolve, 8) PMID 23536288, PMID 23898164, PMID 25345460. While some of these algorithms can successfully identify groups of T cells with shared antigen specificity using single-cell data (TCRdist, GLIPH), and the other algorithms use solely bulk receptor sequencing data (i.e., without access to heavy and light chain sequences), none of these algorithms attempt to approximate the true clonotype for B cells while also attempting to mitigate for sources of noise in the data.

With this understanding of the immune cell's purpose in fighting off attacking foreign antigens, the pharmaceutical industry has strongly focused on developing antibody therapeutics or designing vaccines with the ability to expand antibody lineages directed towards specific B cells with shared antigen specificity. To most effectively determine the efficacy of a vaccine or antibody therapeutic, it is essential to be able to accurately identify cell members of a clonotype, which potentially share common or similar BCRs or antigen specificity. The pharmaceutical industry has also directed its efforts to isolate antibodies and antibody lineages against non-foreign targets for the purpose of developing antibody-based therapeutics for a broad array of disease states including autoimmune disease (anti-inflammatory targets), cancer (checkpoint inhibitors and other targets), and other conditions such as osteoporosis. Similarly, knowing the fine specificities of different antibody lineages elicited by a vaccine is essential to understanding serum neutralization profiles and global epitope maps of an entire virus. This same concept applies to understanding how a patient's adaptive immune system can render drugs such as adalimumab ineffective through the emergence of anti-drug antibodies and distinct anti-drug antibody lineage.

When analyzing a data set of nucleic acid sequence barcode records which includes one or more V(D)J sequence, contigs are assembled for each barcode from the raw sequencing reads. The barcodes for which productive contigs are assembled can be candidates for being called as cell-associated. However, such barcodes need to satisfy a number of filters in order to increase the likelihood of identifying a barcode as cell-associated.

Generally speaking, in a population of clonotypes, it is unlikely to see the same heavy chain in different clonotypes due to the diversity of the heavy chain. This can be addressed, for example, by applying chain-sharing logic in a number of filters (such as, for example, NGRAPH_FILTER, NonDominantJunction, WeakJunction, CommonCloneShado, CommonCloneShadowSingleUmi, and the like) to flag barcodes that have contigs which are not trustworthy and likely belong to another barcode. However, this can result in many false positives, particularly in case of a genetically modified organism carrying BCR or TCR transgenes, such as a transgenic mouse, for example without limitation, a C57BL/6-Tg(IghelMD4)4Ccg/J mouse carrying the IghelMD4 transgene, which recognizes hen egg lysozyme and has high serum titers of anti-lysozyme antibodies, or a C57BL/6(FVB)-Igktm1.1(b12)Nemz Ightm1.1(b12)Nemz/J mouse, which expresses the broadly neutralizing HIV antibody B12.

In a genetically modified organism, for example one carrying only a transgene for a heavy chain, it is expected that there will be a number of clonotypes expressing one transgenic chain and one innate light chain. As a result, the cell population can include legitimate clonotypes that have a shared chain. However, the majority of such barcodes can be filtered out by the application of various filters, such as chain sharing logic filters. This results in recovery of a lower number of cells than were present in the sample, and, from a user perspective, a lower number of cells than expected from the pipeline. Filtering out of barcodes which are incorrectly filtered out due to chain sharing while analyzing data sets including V(D)J sequencing leads to low cell recovery.

As described herein, to overcome the removal of legitimate clonotypes that have a shared chain, and thereby increase recovery of clonotypes, gene expression (GEX) data can be included in the data set which includes V(D)J sequences. For example, in various embodiments in accordance with the present invention, both GEX and V(D)J sequence data can be generated from the same pool of cells, such as from the same cell partitioned in a given well. In some embodiments, the GEX data can be whole transcriptome (e.g., including tens of thousands of genes, or more), whereas the V(D)J data can be generated by selectively amplifying T cell and/or B cell receptor genes.

In certain embodiments, GEX data from the same plurality of single cells can provide a useful signal to distinguish cell-associated partitions from empty partitions, partitions with more than one cell and/or bead, and/or mRNA not associated with a cell, all of which could lead to background signal. Certain filters in the V(D)J library flag “unreliable” barcodes which have V(D)J signal but are not real T cells and/or B cells. In addition to removing most of the artifactual barcode when all the filters are turned on, certain filters can also remove some real cells. While the majority of the artifactual barcodes have low UMI counts in both the GEX and V(D)J sequence data, some barcodes with high numbers of gene expression and V(D)J UMIs can be incorrectly filtered out by the V(D)J pipeline due to chain-sharing. Such barcodes are very likely to be associated with real cells. Due to the common chain between clonotypes (especially in the context of perturbed systems, such as cells sorted from transgenic lines), certain filters (such as, for example, enclone filters) can produce unrealistic barcode grouping. Any clonotype with more than three chains is likely due to a false join. To address this, any clonotype that has more than 3 chains can be split such that each of its constituent exact subclonotypes becomes its own clonotype.

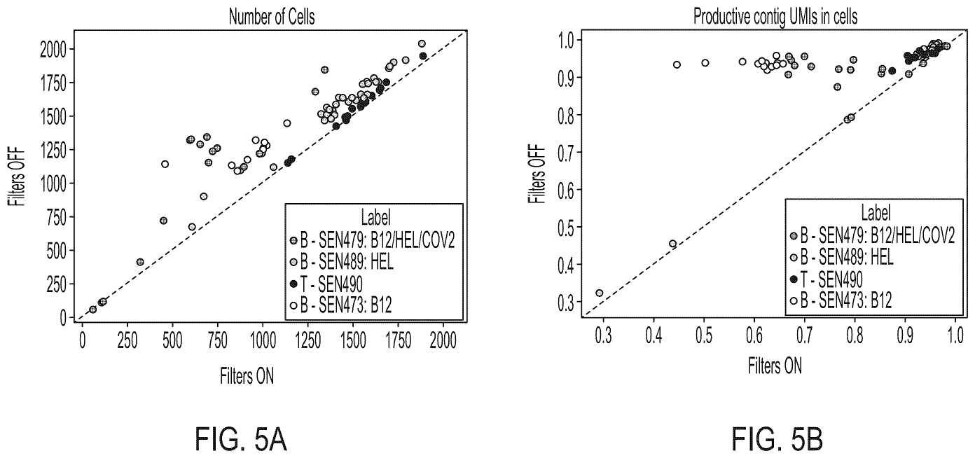

As such, the present inventors have found that retaining only the barcodes in the V(D)J library that are called as cells in the GEX library can eliminate many, or a majority, of unreliable barcodes. When gene expression data is present, the GEX cell calling can be used to filter out many, or a majority, of the unreliable barcodes in the overall data set, which includes V(D)J sequences. The final outputs of the V(D)J analysis are paired receptor sequences at the single cell level, which can be further grouped into clonotypes. Certain filters shown to remove useful data across datasets can be turned off in the presence of gene expression data in order to improve outcomes and increase cell recovery, and V(D)J cell calling. In particular, turning off one or more specific filters as described herein for V(D)J cell calling in the presence of GEX data has been found to result in higher cell recovery. Turning off appropriate V(D)J filters leads to recovery of most of the cell barcodes while still filtering background barcodes. For example, in one exemplary analysis, the number of cells was found to increase by 20%, and the number of usable UMIs increased from 64% to 96%, as described in the exemplary embodiment herein and as shown in FIGS. 5A and 5B. A substantial increase was found to hold across a number of different samples, from a number of different cell types/strains.

Therefore, in accordance with various embodiments, various methods and systems are provided for more effectively identifying paired V(D)J sequences and clonotypes of gene expression data, with higher yield of recovered cells and UMIs than has been possible to date.

### System Embodiment

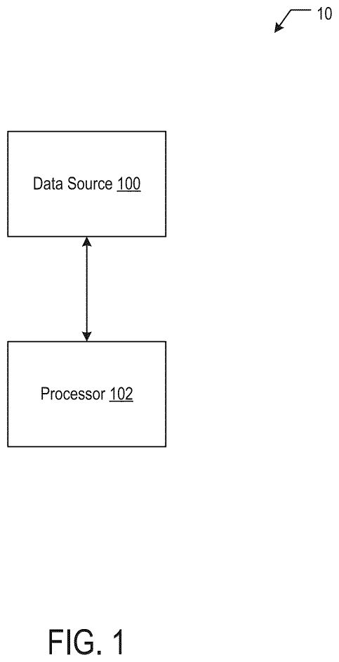

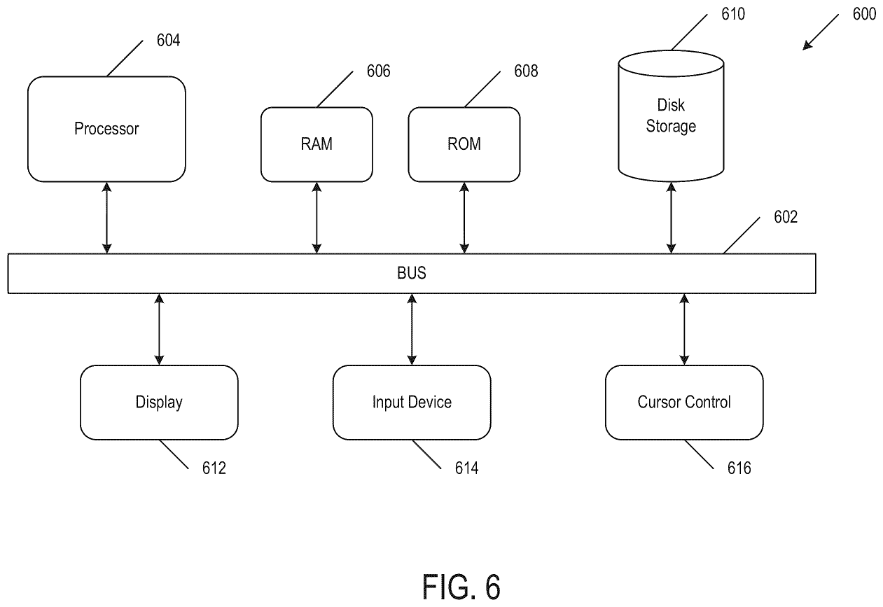

In accordance with various embodiments, FIG. 1 illustrates a block diagram of an example system 10 for analyzing nucleic acid sequences. System 10 comprises a data source 100 and a processor 102. Processor 102 is communicatively connected to data source 100. For example, a user may load data from data source 100 into a software program executed by processor 102. In various embodiments, processor 102 is implemented as an integrated instrument system assembly with data source 100. That is, processor 102 and data source 100 can be housed in the same housing assembly and communicate via conventional device/component connection means (e.g. serial bus, optical cabling, electrical cabling, etc.). For example, data source 100 may be a memory of a computer system (e.g., computer system 600 in FIG. 6) that also includes processor 102.

In other embodiments, processor 102 can be included in a standalone computing device (e.g., computer system 600 in FIG. 6) that is communicatively connected to the data source 100 via an optical, serial port (e.g., USB), network or modem connection. For example, the connection may include one or more wireless networks such as, but not limited to one or more of a Local Area Network (LAN), Wireless Local Area Network (WLAN), a Personal Area Network (PAN), Campus Area Network (CAN), a Metropolitan Area Network (MAN), a Wide Area Network (WAN), a Wireless Wide Area Network (WWAN), Global System for Mobile Communications (GSM), Personal Communications Service (PCS), Digital Advanced Mobile Phone Service (D-Amps), Bluetooth, Wi-Fi, Fixed Wireless Data, 2G, 2.5G, 3G, 4G, LTE networks, enhanced data rates for GSM evolution (EDGE), General packet radio service (GPRS), enhanced GPRS, messaging protocols such as, TCP/IP, SMS, MMS, extensible messaging and presence protocol (XMPP), real time messaging protocol (RTMP), instant messaging and presence protocol (IMPP), instant messaging, USSD, IRC, or any other wireless data networks or messaging protocols. The connection may also include wired networks.

In various embodiments, the functions of processor 102 can be implemented on a distributed network of shared computer processing resources (such as a cloud computing network) that is communicatively connected to data source 100 via a WAN (or equivalent) connection. For example, the functionalities of processor 102 can be divided up to be implemented in one or more computing nodes on a cloud processing service such as AMAZON WEB SERVICES™.

In accordance with various embodiments, data source 100 stores at least one data set comprising a plurality of sequence barcode records from a plurality of cells, e.g., a plurality of immune cells. The data set includes V(D)J sequence information. For instance, each sequence barcode record in the at least one data set comprises at least one V(D)J sequence. In at least one embodiment, the data source 100 stores V(D)J data (e.g., a first data set) and gene expression (GEX) data (e.g., a second data set). In such embodiments, the V(D)J data includes a plurality of sequence barcode records from a plurality of single cells and each sequence barcode record includes at least one V(D)J sequence, and the GEX data includes a plurality of gene expression nucleic acid sequence barcode records from a plurality of single cells. The sequence barcode records in the V(D)J data are from the same plurality of single cells as the gene expression nucleic acid sequence barcode records in the GEX data.

In some embodiments, the data set(s) may include sequence data associated with an immune cell of the plurality of immune cells. In some embodiments, the sequence data associated with the immune cell is generated by at least partitioning a reaction mixture, or a portion thereof, into a plurality of partitions, wherein the reaction mixture comprises (i) a plurality of immune cells, (ii) a target antigen, and optionally (iii) a control antigen, wherein the target antigen is operatively coupled to a first reporter oligonucleotide comprising a first reporter barcode sequence, wherein the control antigen is operatively coupled to a second reporter oligonucleotide comprising a second reporter barcode sequence, and wherein the partitioning provides a partition comprising (i) the immune cell, and (ii) a plurality of nucleic acid barcode molecules comprising a partition-specific barcode sequence; using a first analyte comprising a nucleic acid sequence encoding at least a portion of the antigen binding molecule expressed by the immune cell, and a first nucleic acid barcode molecule of the plurality of nucleic acid barcode molecules, to generate a first barcoded polynucleotide comprising (i) a sequence of the first analyte or reverse complement thereof and (ii) the partition-specific barcode sequence or a reverse complement thereof; and determining a sequence of the first barcoded polynucleotide or derivative thereof.

In some embodiments, the provided partition may further include the target antigen. Exemplary target antigens are described herein. The sequence data associated with the immune cell may further include target antigen data. The target antigen data may be generated by using the first reporter oligonucleotide and a second nucleic acid barcode molecule of the plurality of nucleic acid barcode molecules to generate a second barcoded polynucleotide comprising (i) the first reporter barcode sequence or reverse complement thereof and (ii) the partition-specific barcode sequence or a reverse complement thereof; and determining a sequence of the second barcoded polynucleotide or derivative thereof.

In some embodiments, the provided partition may further include the control antigen. Exemplary control antigens are described herein. The sequence data associated with the immune cell may further include control antigen data. The control antigen data may be generated by using the second reporter oligonucleotide and a third nucleic acid barcode molecule of the plurality of nucleic acid barcode molecules to generate a third barcoded polynucleotide comprising (i) the second reporter barcode sequence or reverse complement thereof and (ii) the partition-specific barcode sequence or a reverse complement thereof; and determining a sequence of the third barcoded polynucleotide or derivative thereof.

In some embodiments, the sequence dataset associated with the immune cell may further include data generated by using a second analyte comprising a nucleic acid sequence encoding at least a different portion of the antigen binding molecule expressed by the immune cell and a fourth nucleic acid barcode molecule of the plurality of nucleic acid barcode molecules to generate a fourth barcoded polynucleotide comprising (i) a sequence of the second analyte or reverse complement thereof and (ii) the partition-specific barcode sequence or a reverse complement thereof, and determining a sequence of the fourth barcoded polynucleotide or derivative thereof.

In some embodiments, the first analyte may include one or more of a variable (V) gene segment sequence, a joining (J) gene segment sequence, a diversity (D) gene segment sequence, or a constant (C) gene segment sequence of the antigen binding molecule.

In some embodiments, the second analyte may include one or more of a variable (V) gene segment sequence, a joining (J) gene segment sequence, a diversity (D) gene segment sequence, or a constant (C) gene segment sequence of the antigen binding molecule.

In some embodiments, the first analyte may encode at least a portion of a B cell receptor (BCR) heavy chain. The second analyte may encode at least a portion of a B cell receptor (BCR) light chain of the antigen binding molecule.

In some embodiments, the first analyte may encode at least a portion of a T cell receptor (TCR) alpha chain. The second analyte may encode at least a portion of a T cell receptor (TCR) beta chain of the antigen binding molecule.

In some embodiments, any one or more of the first, second, third, and fourth barcoded polynucleotides may include a unique molecular identifier (UMI) sequence or a reverse complement thereof.

In some embodiments, the sequence data associated with the immune cell includes a UMI Count/antigen, e.g., a target antigen UMI count. The target antigen UMI count may be determined based at least on a quantity of unique molecular identifier (UMI) sequences or reverse complements of unique molecular identifier (UMI) sequences associated with (i) the partition-specific barcode sequence or a reverse complement thereof, and (ii) the first reporter barcode sequence or a reverse complement thereof.

In some embodiments, the sequence data associated with the immune cell includes a control antigen UMI count. The control antigen UMI count may be determined based at least on a quantity of unique molecular identifier (UMI) sequences or reverse complements of unique molecular identifier (UMI) sequences associated with (i) the partition-specific barcode sequence or a reverse complement thereof, and (ii) and the second reporter barcode sequence or a reverse complement thereof.

In some embodiments, the sequence data associated with the immune cell includes an antigen specificity determination. The antigen specificity determination may be based on the target antigen UMI count and the control antigen UMI count.

A target antigen can be, e.g., a user-selected antigen for which binding by an antigen binding molecule, e.g., an antibody, BCR, or antigen binding fragment thereof, is desired. In some embodiments, the target antigen can include a target antigenic peptide, bound to an MHC molecule, to which binding by a TCR or antigen binding fragment thereof is desired. The target antigen can be associated with an infectious agent such as a viral, bacterial, parasitic, protozoal or prion agent. The target antigen may be an antigen associated a viral agent. In these instances, the viral agent may be an influenza virus, a coronavirus, a retrovirus, a rhinovirus, or a sarcoma virus. In other instances, the viral agent may be severe acute respiratory syndrome coronavirus 1 (SARS-CoV-1), a SARS-CoV-2, a Middle East respiratory syndrome coronavirus (MERS-CoV), or human immunodeficiency virus (HIV), influenza, respiratory syncytial virus, or Ebola virus. The target antigen may be an antigen associated with a tumor or a cancer. Antigens associated with a tumor or cancer, include any of epidermal growth factor receptor (EGFR), CD38, platelet-derived growth factor receptor (PDGFR) alpha, insulin growth factor receptor (IGFR), CD20, CD19, CD47, ERBB2IP, TP53, KRAS, MAGEA1, LC3A2, KIAA0368, CADPS2, CTSB or human epidermal growth factor receptor 2 (HER2). The target antigen may be an checkpoint molecule associated with tumors or cancers (e.g., CD38, PD-1, CTLA-4, TIGIT, LAG-3, VISTA, TIM-3), or it may be a cytokine, a GPCR, a cell-based co-stimulatory molecule, a cell-based co-inhibitory molecule, an ion channel, or a growth factor. The target antigen may be associated with a degenerative condition or disease.

In some embodiments, a control antigen is a non-target antigen (e.g., negative control antigen). Non-target antigens (e.g., negative control antigens) may be any antigen to which the antibodies or antigen-binding fragments thereof, would not be expected to bind. In some embodiments, the non-target antigen has been selected such that it is not expected to bind the antibody or antigen-binding fragment thereof. By way of example, the non-target antigen may be any antigen for which a subject (e.g., a human subject) would not be expected to develop an antibody response to or to have antibodies with a specificity for. Such a non-target antigen may be an antigen endogenous to and abundantly expressed in a subject, e.g., a human subject, e.g., human serum albumin (HSA).

In some embodiments wherein the target antigen is a target MHC molecule complex comprising a target antigenic peptide bound to an MHC molecule, the control antigen may be a control MHC molecule complex comprising a control peptide bound to an MHC molecule. The control peptide may be a scrambled peptide, serum albumin peptide, a heteroclitic peptide, or peptide to which immune cells of the sample are naïve. The scrambled peptide may have the same amino acid residue composition as a target antigenic peptide (bound to the first MHC molecule of the target MHC molecule complex), wherein the amino acid residues are presented in a different, e.g., scrambled, order relative to that of the target antigenic peptide. The serum albumin peptide may be a human or mouse serum albumin peptide. The control peptide may be any peptide, e.g., not only a serum albumin peptide, to which the ABMs of the plurality of immune cells would not be expected to bind, e.g., cardiolipin, keyhole limpet hemocyanin, flagellin or insulin. In instances in which the control peptide is a peptide to which ABMs of the plurality of immune cells would not be expected to bind, the control peptide may be a peptide of an abundantly expressed self-antigen of a subject from which the plurality of immune cells had been obtained. In other instances in which the control peptide is a peptide to which ABMs of the plurality of immune cells would not be expected to bind, the control peptide may be a peptide or peptide fragment of an antigen to which the plurality of immune cells are naïve. For example, the control peptide may be a peptide or peptide fragment of an antigen of a virus, e.g. HIV (e.g., TPGPGVRYPL), if the subject from which the plurality of immune cells have been obtained, has not been exposed to the virus, e.g., HIV. For other example, the control peptide may be a heteroclitic peptide. Heteroclitic peptides may include peptides having valine, or leucine or other suitable residues at positions that anchor the peptide to the second MHC molecule, e.g., position 2 and/or a C-terminal residue, but alanine residues at the remaining amino acid positions (e.g., ALAAAAAAV, ATAAAAAAK, AYAAAAAAL, APAAAAAAV or RYAAAAALL). Additional examples of negative control peptides include ASYAAAAV and vaccinia virus peptide TSYKFESV.

In some embodiments, the sequence data associated with the immune cell includes gene expression data. In some embodiments, the sequence data associated with the immune cell includes whole transcriptome data.

Processor 102 can apply various filters to the data set(s) stored in data source 100 (e.g., when the data set is uploaded to a software program executed by processor 102) in order to identify one or more V(D)J cells. In some embodiments, processor 102 may group the identified V(D)J cells into one or more clonotypes. As will be described further below, the filters applied may vary based on whether gene expression data is included in the data set(s). The filtered data including the identified V(D)J cells and/or clonotypes can then be output (e.g., to a user interface of a display) for user review.

### Method Embodiment

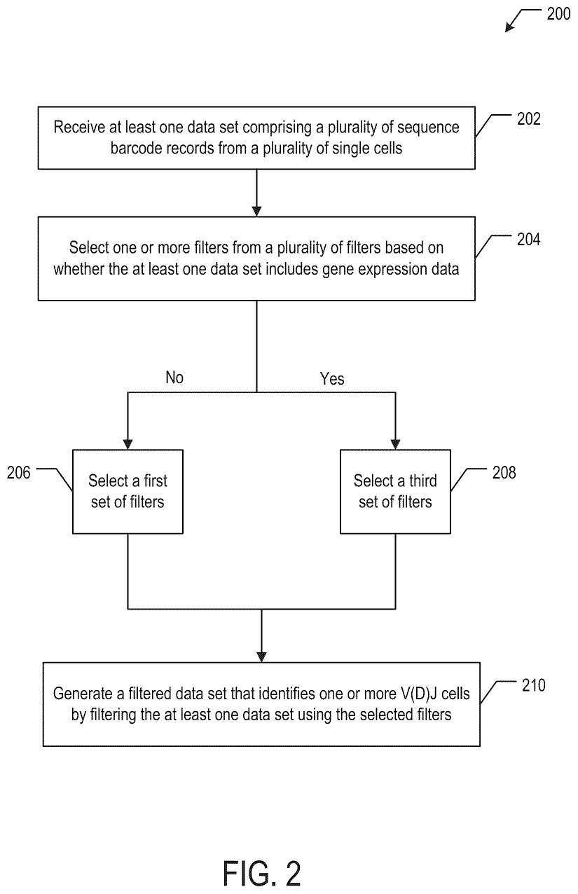

In accordance with various embodiments, an example method 200 for identifying one or more V(D)J cells from at least one data set including a plurality of sequence barcode records is illustrated in FIG. 2. At block 202, at least one data set is received. For example, processor 102 may receive the at least one data set from data source 100 when a user manually uploads the at least one data set from data source 100 to software executed by processor 102. The at least one data set includes a plurality of sequence barcode records (e.g., barcodes) from a plurality of cells, e.g., a plurality of immune cells. The data set includes V(D)J sequence information. For instance, each sequence barcode record in the at least one data set comprises at least one V(D)J sequence.

At block 204, one or more filters are selected from a plurality of filters based on whether the at least one data set includes gene expression data (e.g., gene expression nucleic acid sequence barcode records). In some embodiments, method 200 includes detecting whether the at least one data set includes gene expression data. For example, processor 102 may automatically identify gene expression data in the received at least one data set. In other embodiments, a user may manually indicate whether the at least one data set being provided includes gene expression data, such as by checking a box or toggling a switch when uploading the at least one data set. It will be appreciated that processor 102 receiving a manual indication from a user regarding whether the at least one data set includes gene expression data can also be considered as processor 102 detecting whether the at least one data set includes gene expression data.

At block 206, if gene expression (GEX) data is absent from the at least one data set, then a first set of filters is selected. The first set of filters includes a second set of filters (e.g., GEX data dependent filters) that are configured to eliminate sequence barcode records that comprise an identical V(D)J sequence as that of another sequence barcode record. In non-limiting embodiments, the at least one data set may include a first sequence barcode record and a second sequence barcode record. The second set of filters are configured to remove the first sequence barcode record from the at least one data set when the first and second sequence barcode records include an identical V(D)J sequence. As used in the context of the second set of filters, the term “identical” means that at least 100% of the V(D)J sequence of the first sequence barcode is the same as the V(D)J sequence of the second sequence barcode. In one example, the first set of filters includes all of the plurality of filters. In non-limiting embodiments, in this example, when GEX data is absent from the at least one data set, all of the filters are selected. Further information regarding the filters included in the first and second sets of filters is provided below in the Filters subsection of the Additional Features section. For example, the available filters include initial filters, chain sharing-based filters, and single cell filters, and a subset of these available filters are GEX data dependent.

At block 208, if gene expression (GEX) data is present in the at least one data set, then a third set of filters is selected. The third set of filters includes the first set of filters minus the second set of filters. In non-limiting embodiments, the second set of filters are not selected because GEX data is included in the at least one data set, but the remaining filters of the first set of filters are selected and included in the third set of filters. Selecting filters may include activating and/or deactivating certain filters. For example, in some embodiments, all of the filters in the first set of filters may be active by default. In such embodiments, the second set of filters may be deactivated when GEX data is present in the at least one data set. In other example embodiments, all of the filters in the first set of filters may be inactive by default. In such embodiments, the second set of filters remain inactive, while the remaining filters in the first set of filters are activated when GEX data is present in the at least one data set.

At block 210, a filtered data set is generated that identifies one or more V(D)J cells from the plurality of cells by filtering the at least one data set using the selected filters.

While method 200 of FIG. 2 illustrates one example method for identifying one or more V(D)J cells from at least one data set including a plurality of sequence barcode records, it should be noted that various methods for identifying one or more V(D)J cells from a data set including a plurality of sequence barcode records are contemplated herein, and can include various combinations of blocks discussed herein. For example, the order of some of the blocks may be changed, certain blocks may be combined with other blocks, and some of the blocks described are optional. The method 200 may be performed by processing logic that may comprise hardware (circuitry, dedicated logic, etc.), software, or a combination of both. This applies to a non-transitory computer-readable medium, as well, in which instructions are stored, which when executed by a processor, cause the processor to perform operations detailed above for identifying one or more V(D)J cells from at least one data set including a plurality of sequence barcode records, as discussed herein. This further applies to systems for identifying one or more V(D)J cells from at least one data set including a plurality of sequence barcode records, as discussed herein.

In accordance with various embodiments of the methods for identifying one or more V(D)J cells, the at least one data set comprises a first data set (e.g., V(D)J data) and a second data set (e.g., GEX data), and wherein the sequence barcode records of the second data set comprise a plurality of gene expression nucleic acid sequence barcode records such that gene expression data is present in the at least one data set.

In accordance with various embodiments of the methods for identifying one or more V(D)J cells, each gene expression nucleic acid sequence barcode record comprises at least one cDNA sequence.

In accordance with various embodiments of the methods for identifying one or more V(D)J cells, the sequence barcode records of the first data set (e.g., V(D)J data) are from the same plurality of single cells as the sequence barcode records of the second data set (e.g., GEX data).

In accordance with various embodiments, the various methods for identifying one or more V(D)J cells can further include generating, with the computing device, a modified second data set (e.g., modified GEX data) by determining sequence barcode records of the plurality of gene expression nucleic acid sequence barcode records in the second data set that are associated with a single cell of the plurality of single cells.

In accordance with various embodiments of the methods for identifying one or more V(D)J cells, the plurality of gene expression nucleic acid sequence barcode records include data associated with a plurality of partitions that each include a single cell and data associated with partitions that include two or more cells, and wherein generating the modified second data set (e.g., modified GEX data) comprises removing from the second data set (e.g., GEX data) the data associated with the partitions that include two or more cells.

In accordance with various embodiments of the methods for identifying one or more V(D)J cells, the third set of filters includes a first subset of filters (e.g., initial filters) and a second subset of filters (e.g., single cell filters except for those single cell filters that are GEX data dependent), and wherein generating the filtered data set comprises: generating, with the computing device, a third data set by filtering the sequence barcode records of the first data set (e.g., V(D)J data) using the first subset of filters; generating, with the computing device, a fourth data set by removing from the third data set any sequence barcode record in the third data set that is absent from the modified second data set (e.g., modified GEX data); and generating, with the computing device, the filtered data set by filtering the fourth data set using the second subset of filters.

In accordance with various embodiments of the methods for identifying one or more V(D)J cells, the first subset of filters (e.g., initial filters) are configured to remove sequence barcode records from the first data set (e.g., V(D)J data) that have a transcript expression level below a threshold.

In accordance with various embodiments of the methods for identifying one or more V(D)J cells, the second subset of filters (e.g., single cell filters excluding those single cell filters that are GEX data dependent and turned off) comprises a plurality of filters configured to remove from the fourth data set sequence barcode records that contain material from two or more cells, cell fragments, or individual mRNA molecules.

In accordance with various embodiments, the various methods for identifying one or more V(D)J cells can further include generating, with the computing device, a modified filtered data set by grouping the one or more V(D)J cells in the filtered data set into at least one clonotype. Each clonotype consists of all descendants of a single, fully rearranged common ancestor, as approximated computationally.

In accordance with various embodiments, the various methods for identifying one or more V(D)J cells can further include identifying, with the computing device and from the modified filtered data set, a clonotype having a quantity of chains greater than a predetermined threshold, the clonotype including a plurality of subclonotypes; and replacing the identified clonotype with each of the plurality of subclonotypes such that each subclonotype becomes a respective clonotype.

In accordance with various embodiments of the methods for identifying one or more V(D)J cells, each of the one or more V(D)J cells are a T cell and/or a B cell.

In accordance with various embodiments, the various methods for identifying one or more V(D)J cells can further include detecting, with the computing device, whether the at least one data set includes gene expression data.

In accordance with various embodiments, of the methods for identifying one or more V(D)J cells, the second set of filters includes a filter (e.g., the ninth or eleventh filter described below) configured to remove a sequence barcode record based on a unique molecular identifier (UMI) count of the sequence barcode record.

Workflow without GEX Data

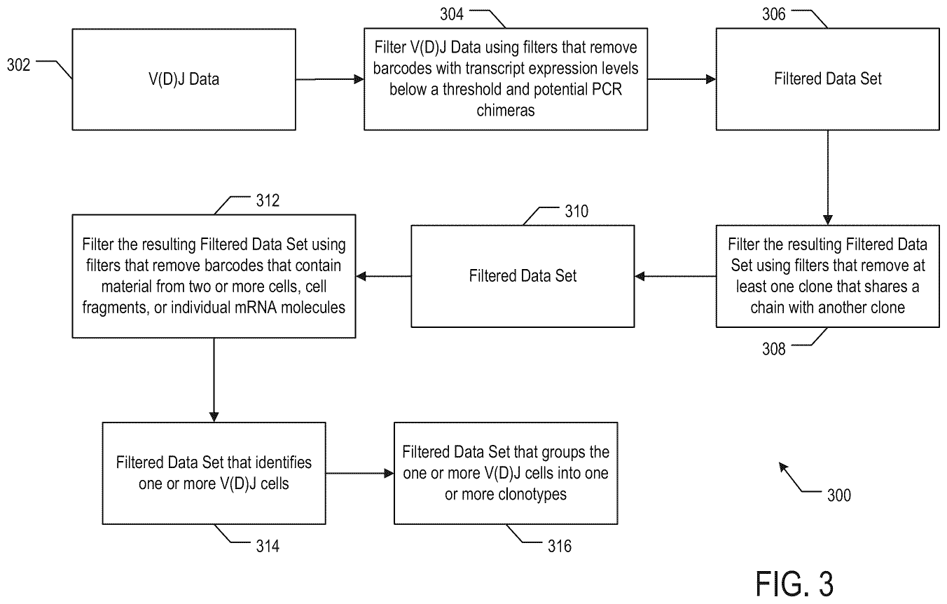

In accordance with various embodiments, an example workflow 300 for identifying one or more V(D)J cells is illustrated in FIG. 3. The example workflow 300 may be performed to identify one or more V(D)J cells from V(D)J data. In non-limiting embodiments, the example workflow 300 is performed to find the set of barcodes in the V(D)J data that contain a T or B cell and filter out the remaining barcodes. In comparison to the following section (e.g., workflow 400), the workflow 300 does not include gene expression (GEX) data. It will be appreciated that all aspects of the example method 200 that are applicable to filtering V(D)J data in the absence of gene expression (GEX) data are also applicable to the workflow 300.

In various embodiments, the workflow 300 begins with receiving V(D)J data 302. In some aspects, the V(D)J data 302 is generated by determining if received V(D)J data is a T cell enrichment or a B cell enrichment and the reads in the V(D)J data are subsequently sorted by barcode to generate the V(D)J data 302. Sorting the reads in the V(D)J data by barcode, in such aspects, includes piecing the reads together to output a set of assembled contigs that represent the best estimate of V(D)J transcript sequences in the V(D)J data. As such, the V(D)J data 302 is a set of barcodes including at least one productive full-length V(D)J transcripts per barcode, and optionally including paired heavy/light or alpha/beta V(D)J transcripts.

At block 304, the V(D)J data 302 is filtered using a first set of filters (e.g., initial filters) to generate a filtered data set 306. The initial filters are configured to remove barcodes from the V(D)J data 302 with transcript expression levels below a threshold and potential PCR chimeras.

At block 308, the filtered data set 306 is filtered using a second set of filters (e.g., chain sharing-based filters) to generate a filtered data set 310. The chain sharing-based filters remove noise introduced by cells containing large amounts of RNA (e.g., typically plasma B cells). Specifically, the chain sharing-based filters are configured to remove a clone that shares a chain with another clone. For instance, the chain sharing-based filters are configured to remove low-frequency clones that share a chain with a higher-frequency or large clone, and remove high-frequency clones to remove noise from mRNA leakage caused by sample processing (e.g., not due to biological clonal expansion). In this context, frequency refers to the number of barcodes with the same V(D)J transcript.

At block 312, the filtered data set 310 is filtered using a third set of filters (e.g., single cell filters) to generate a filtered data set 314. The single cell filters are configured to remove barcodes that contain material from two or more cells, cell fragments, or individual mRNA molecules so that barcodes that contain material from single cells remain. The barcodes that survive the various filtering steps in workflow 300 and remain in the filtered data set 314 are identified as V(D)J cells that have one or more full-length productive V(D)J contigs.

In various embodiments, the barcodes of the identified V(D)J cells in the filtered data set 314 are grouped into one or more clonotypes to generate a filtered data set 316 (e.g., a modified filtered data set 314). Each clonotype consists of all descendants of a single, fully rearranged common ancestor, as approximated computationally. The filtered data set 314 and/or the filtered data set 316 may be presented to a user for review.

In accordance with various embodiments of the workflow 300, each of the one or more V(D)J cells are a T cell and/or a B cell.

### Workflow with GEX Data

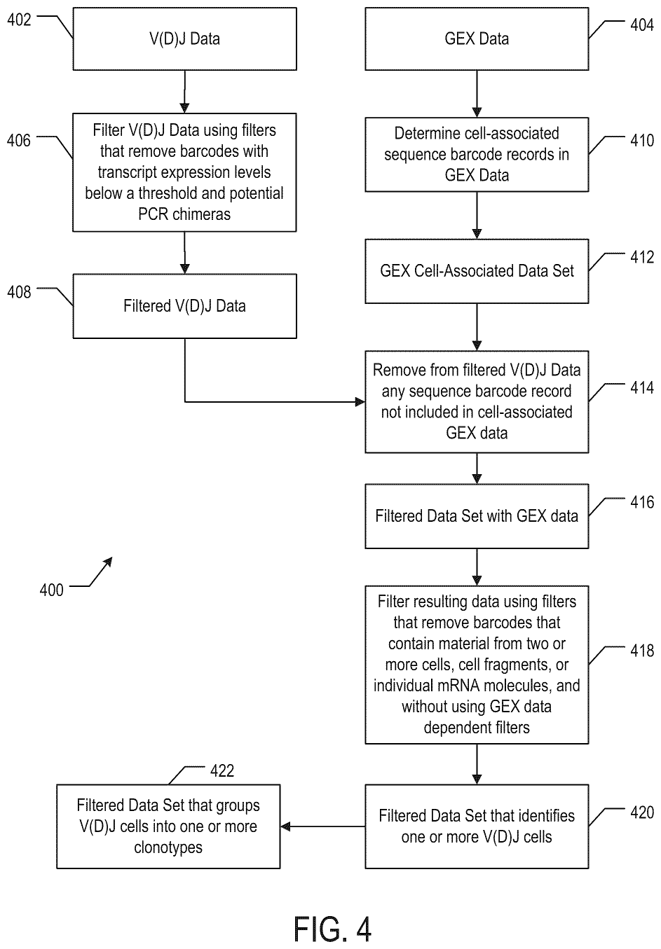

In accordance with various embodiments, an example workflow 400 for identifying one or more V(D)J cells is illustrated in FIG. 4. The example workflow 400 may be performed to identify one or more V(D)J cells from V(D)J data analyzed together with gene expression (GEX) data. In non-limiting embodiments, the example workflow 400 is performed to find, with the help of GEX data, the set of barcodes in the V(D)J data that come/originate from a single T cell receptor (TCR) or single B cell receptor (BCR) cell and filter out the remaining barcodes. In comparison to the preceding section (e.g., the workflow 300) which did not include GEX data, the workflow 400 includes GEX data. It will be appreciated that all aspects of the example method 200 that are applicable to filtering V(D)J data in the presence of gene expression (GEX) data are also applicable to the workflow 400.

In various embodiments, the workflow 400 includes receiving V(D)J data 402 (e.g., a first data set). In some aspects, the V(D)J data 402 is generated by determining if received V(D)J data is a T cell enrichment, a B cell enrichment and/or a combination thereof. The barcode reads in the V(D)J data are sorted by barcode to generate the V(D)J data 402. Sorting the reads in the V(D)J data by barcode, in such aspects, includes piecing the reads together to output a set of assembled contigs that represent the best estimate of V(D)J transcript sequences in the V(D)J data. As such, the V(D)J data 402 is a set of barcodes from a plurality of single cells, the barcodes including at least one productive full-length V(D)J transcripts per barcode, and optionally including paired heavy/light or alpha/beta V(D)J transcripts.

The workflow 400 also includes receiving gene expression (GEX) data 404 (e.g., a second data set). The GEX data 404 is a set of GEX nucleic acid sequence barcode records from a plurality of single cells. The set of GEX nucleic acid sequence barcode records include at least one productive full-length V(D)J transcripts per barcode. In at least some embodiments, each GEX nucleic acid sequence barcode record comprises at least one cDNA sequence.

At block 406, the V(D)J data 402 is filtered using a first set of filters (e.g., initial filters) to generate a filtered V(D)J data set 408 (e.g., a third data set). The initial filters are configured to remove barcodes from the V(D)J data 402 with transcript expression levels below a threshold and potential PCR chimeras.

At block 410, in at least some embodiments, GEX reads in the GEX data 404 are aligned to a reference. Barcodes associated with a single cell of the plurality of single cells are then determined from the GEX data 404 and grouped to generate a GEX cell-associated data set 412. For example, the GEX data 404 includes data associated with a plurality of partitions wherein each partition comprises a single cell, and data associated with partitions comprising two or more cells, free floating mRNA which leaked from cells, or no cell at all. The data associated with partitions comprising two or more cells, free floating mRNA, or no cell is removed from the GEX data 404 in order to generate the GEX cell-associated data set 412 which includes only barcode records that are from a single cell in a partition.

At block 414, a filtered data set 416 with GEX data (e.g., fourth data set) is generated by removing barcodes from the V(D)J data set 408 that are not included in the GEX cell-associated data set 412.

At block 418, the filtered data set 416 with GEX data is filtered using a second set of filters (e.g., single cell filters excluding GEX data dependent filters) to generate a filtered data set 420 (e.g., fifth data set) that identifies one or more V(D)J cells. For example, the second set of filters may include the twelfth filter, thirteenth filter, fifteenth through the twentieth filters, and twenty-third through the twenty-eighth filters described below in the Filters subsection of the Additional Features section. The single cell filters are configured to remove barcodes that contain material from two or more cells, cell fragments, or individual mRNA molecules so that barcodes that contain material from single cells remain. In some aspects, the second set of filters at block 418 includes at least one chain sharing-based filter when the ninth filter described in the filters section below is treated as a chain-sharing based filter rather than an initial filter. The barcodes that survive the various filtering steps in workflow 400 and remain in the filtered data set 420 are identified as V(D)J cells that have one or more full-length productive V(D)J contigs.

In various embodiments, the barcodes of the identified V(D)J cells in the filtered data set 420 are grouped into one or more clonotypes to generate a filtered data set 422 (e.g., a modified filtered data set 420). Each clonotype consists of all descendants of a single, fully rearranged common ancestor, as approximated computationally. The filtered data set 420 and/or the filtered data set 422 may be presented to a user for review.

In some aspects of these embodiments in which V(D)J data is analyzed together with gene expression (GEX) data, while conducting the filtering operations without the GEX data dependent filters can result in better cell recovery, having these GEX data dependent filters inactive can, in some instances, result in unrealistic clonotype grouping. One of the main reasons such unrealistic grouping can happen when filtering without the GEX data dependent filters is that the existence of shared chains between cells that belong to different clonotypes can affect the filtering. As such, to address the unrealistic clonotype grouping, in some embodiments, at least one clonotype may be split into the subclonotypes of the clonotype if the clonotype has a quantity of chains greater than a predetermined threshold (e.g., three chains). In non-limiting embodiments, the clonotype is replaced by each of its subclonotypes such that each subclonotype is treated as a respective clonotype. The reason for this clonotype splitting in these embodiments is that it's biologically extremely unlikely to have an immune cell with more than three chains. As such, clonotypes that contain more than three chains are most likely because of incorrect grouping. Conversely, the grouping at exact subclonotype level may be used because cells within an exact subclonotype have the same number of chains and the same sequence for each of the chains.

In accordance with various embodiments of the workflow 400, each of the one or more V(D)J cells are a T cell and/or a B cell.

In summary, and in accordance with various embodiments herein, the systems and methods exemplified herein solve multiple problems, including identifying one or more V(D)J cells from at least one data set that can include a massive amount of sequence barcode records. Additionally, the systems and methods exemplified herein solve the problem of gene expression data affecting the filtering process used when gene expression data is absent for identifying one or more V(D)J cells from at least one data set.

### Example Implementation

In accordance with various embodiments, the effect of the gene expression (GEX) data dependent filters was shown for a transgenic B12 mouse model.

GEX and V(D)J sequence data were collected for mouse cells from the C57BL/6(FVB)-Igk^{tm1.1(b12)Nemz} Igh^{tm1.1(b12)Nemz}/J strain, which is a b12HL mutant mouse strain expressing the broadly neutralizing HIV antibody b12. After gene expression sequencing to obtain whole transcriptome data, 1444 cells were recovered and retained for the pipeline. From these cells, 3045 sequence barcodes were analyzed and found to have a V(D)J signal (e.g. a productive contig).

Because not all barcodes with V(D)J signal are V(D)J cells, the data set was subjected to a number of filters to screen out sequence barcodes which were not truly associated with a T cell and/or B cells. After filtering, 1022 V(D)J cells were recovered (70%) among the 1444 cells for which GEX data was obtained.

Turning off certain V(D)J filters increased the number of recovered V(D)J cells from 1022 (70%) to 1279 (89%). Therefore, turning off the certain V(D)J filters allowed for recovery of most of the cell barcodes while still filtering background barcodes. In this sample, the number of cells increased by 20%, and the number of usable UMIs increased from 64% to 96%.

This is shown in FIGS. 5A and 5B, which depict the increased number of V(D)J cells recovered and number of productive contig UMIs, respectively, when certain filters are turned off in the presence of GEX data. The x-axis indicates filters turned on, and the y-axis indicates the number after turning off the relevant filters.

FIG. 5A is a graph showing the number of V(D)J cells recovered when the GEX data dependent filters were off (i.e. not used) compared to when the GEX data dependent filters were on (i.e. used).

FIG. 5B is a graph showing the number of productive contig UMIs identified in cells when the gene expression (GEX) data dependent filters were off (i.e. not used) compared to when the GEX data dependent filters were on (i.e. used).

### Additional Features

### Filters

As described above, various filters of a plurality of filters are used to filter a at least one data set in order to identify one or more V(D)J cells from the at least one data set. These filters may be configured to remove sequence barcode records from the data set that meet a variety of criteria. Examples of filters that may be used are described in the remaining portion of this section. As used herein, a set of filters can, in some instances, include a single filter.

In various embodiments, the plurality of filters include some or all of the filters included in a first set of filters (e.g., initial filters). The initial filters are configured to remove sequence barcode records from a data set that have a transcript expression level below a threshold.

The first set of filters includes a first filter configured to remove a sequence barcode record (e.g., barcode) that has less than a predetermined threshold of surviving non-solo unique molecular identifiers (UMIs). A surviving UMI can be determined by: (i) finding strong paths in a graph that have an annotation or match to a primer; (ii) finding all the edges of a strong path; (iii) finding all the reads on such a good edge; (iv) finding the UMIs for these reads; and (v) restricting to those UMIs for which 50% of their kmers are contained in good edges. In certain aspects, if no strong path had a V annotation, then all surviving units are killed. A non-solo UMI is defined as a UMI that has more than one read or the N50 reads per UMI is two.

The first set of filters includes a second filter configured to remove a sequence barcode record that has less than a predetermined threshold of surviving non-solo UMIs and has less than a predetermined threshold of UMIs.

The first set of filters includes a third filter configured to remove a sequence barcode record that does not include a contig having a V annotation.

The first set of filters includes a fourth filter configured to remove a sequence barcode record that includes exactly one productive contig and includes a junction support contig.

The first set of filters includes a fifth filter configured to remove a sequence barcode record that does not include a confident contig.

The first set of filters includes a sixth filter configured to remove a sequence barcode record that has a maximum number of reads among the surviving UMIs that is less than 3% of the N50 reads per UMI computed for each barcode.

The first set of filters includes a seventh filter configured to remove a sequence barcode record having a contig that is included in a set of contigs sharing the same junction segment when the following criteria is satisfied: (i) the number of contigs in the set is less than a first predetermined threshold; (ii) the median UMIs supporting the junction segment is less than a second predetermined threshold; (iii) the maximum UMIs supporting the junction is greater than or equal to a heuristic parameter/threshold. These sequence barcode record are removed because they appear to arise from some sort of leakage from plasma cells.

The first set of filters includes an eighth filter configured to remove a first sequence barcode record of a pair of sequence barcode records that share the same junction when the first sequence barcode record satisfies the following criteria: (i) junction UMIs for the second sequence barcode record is greater than a predetermined threshold, (ii) junction UMIs for the first sequence barcode record is equal to one, (iii) the second sequence barcode record has greater than or equal to two high confidence contigs, (iv) the first sequence barcode record has greater than or equal to three high confidence contigs, and (v) there is only one common chain between the first and second sequence barcode records allowing for some mutation.

In various embodiments, the plurality of filters include some or all of a second set of filters (e.g., chain sharing based filters). The chain sharing-based filters remove noise introduced by cells containing large amounts of RNA (e.g., typically plasma B cells). Specifically, the chain sharing-based filters are configured to remove a clone that shares a chain with another clone. For instance, the chain sharing-based filters are configured to remove low-frequency clones that share a chain with a higher-frequency or large clone, and remove high-frequency clones to remove noise from mRNA leakage caused by sample processing (e.g., not due to biological clonal expansion). In this context, frequency refers to the number of barcodes with the same V(D)J transcript. In at least some aspects, the filters included in the first set are applied prior to the filters included in the second set when filtering a at least one data set to identify one or more V(D)J cells.

The second set of filters includes a ninth filter configured to remove a sequence barcode record based on V segments that appear in a set of contigs sharing the same CDR3 nucleotide. For instance, consider the V segments that appear in a set of contigs sharing the same CDR3 nucleotide. If any V segment has collective UMI support (i.e. sum of UMIs across all sequence barcode records) at least a threshold amount greater than another V segment, then the sequence barcode records containing weaker contigs are removed. In some aspects, this ninth filter is included in the first set of filters instead of the second set of filters.

The second set of filters includes a tenth filter configured to remove a sequence barcode record based on a set of junctions associated with two or more high confidence contigs. For instance, consider a sequence barcode record having two or more high confidence contigs. If (1) a number of sequence barcode records in which the same set of junctions is observed is less than or equal to a first predetermined threshold, and (2) the maximum number of sequence barcode records that share a given set of two or more high confidence contigs, where at least one contig has a junction in the set of junctions associated with the two or more high confidence contigs, is greater than or equal to a second predetermined threshold multiplied by the number of sequence barcode records in which the same set of junctions is observed, then the sequence barcode record having two or more high confidence contigs is removed. In some embodiments, if a sequence barcode record has exactly two high confidence contigs, SHMs are accounted for by ensuring that non-matching junctions differ in at least ten bases.

The second set of filters includes an eleventh filter configured the same as the tenth filter except that the eleventh filters is only applicable to sequence barcode records with exactly two high confidence contigs and having only a single UMI supporting the junction of one of the two contigs. SHMs are not accounted for in regards to the eleventh filter.

In various embodiments, the plurality of filters includes some or all of a third set of filters (e.g., single cell filters). The single cell filters are configured to remove barcodes that contain material from two or more cells, cell fragments, or individual mRNA molecules so that barcodes that contain material from single cells remains. In at least some embodiments, the filters in the third set of filters are applied in the order in which they are listed in the following paragraphs when filtering a at least one data set to identify one or more V(D)J cells.

The third set of filters includes a twelfth filter configured to remove sequence barcode records not called cells in a pipeline for processing and transforming sequencing data into the at least one data set. For example, a software tool that processes and inputs sequencing data for producing the at least one data set within the disclosure can be the Cell Ranger™ Software. In such an example, the twelfth filter is configured to remove sequence barcode records not called cells in the V(D)J pipeline of the Cell Ranger™ Software.

The third set of filters includes a thirteenth filter configured to remove sequence barcode records having more than four productive contigs.

The third set of filters includes a fourteenth filter configured to remove some exact subclonotypes that appear to be background mRNA or a phenotypically similar phenomenon by virtue of their relationship to other exact subclonotypes.

The third set of filters includes a fifteenth filter configured to remove spurious exact subclonotypes using cross-library information. For instance, if one specifies that two or more libraries arose from the same sample (i.e., from the same tube of cells), then expanded exact subclonotypes that are present in one library but not another, in a fashion that would be highly improbable, assuming random draws of cells from the tube, can be removed. Such observed behavior can be understood to arise when a plasma or plasmablast cell breaks up during or after pipetting from the tube, and the resulting fragments seed can yield ‘fake’ cells.

The third set of filters includes a sixteenth filter configured to remove duplicated sequence barcode records within an exact subclonotype.

The third set of filters includes a seventeenth filter configured to remove rare artifacts arising from gel bead contamination.

The third set of filters includes an eighteenth filter configured to remove some four-chain clonotypes that might represent doublets. For example, the eighteenth filter may remove a foursie exact subclonotype that contains a twosie exact subclonotype having at least ten cells, no matter how many cells the foursie exact subclonotype has. The foursies that are killed are believed to be rare odd artifacts arising from repeated cell doublets or, for example, GEMs (gel bead in emulsion) that contain two cells and multiple gel beads.

The third set of filters includes a nineteenth filter configured to remove exactly subclonotypes having multiple chains that are all of the same type.

The third set of filters includes a twentieth filter configured to disintegrate some single-chain clonotypes into single cells.

The third set of filters includes a twenty-first filter configured to remove some B cells having UMI counts below a threshold. For example, the threshold may be determined as follows. For any cell, “umitot” is defined as the sum of the maximum UMI count for a cell's heavy chains (if any) and the maximum UMI count for the cell's light chains (if any). A baseline value is then established for the expected value of “umitot”, for each data set taken individually. To do this, all clonotypes having exactly one cell and exactly one heavy and light chain each are examined. If there are less than 20 such cells, the twenty-first filter is not applied to cells in that data set. Otherwise, let n_50% denote the median of the “umitot” values for the data set and let n_10% denote the 10^{th} percentile of the “umitot” values for the data set. The threshold is calculated as min(n_10%, n_50%−4*sqrt(n_50%)). Next, from each clonotype having at least two cells, every cell that has a UMI count below the threshold is removed with the following qualifications. Let k be the number of cells to be removed in a clonotype having n cells. Then, for a binomial distribution having p=0.1, it is required that the probability of observing k or more events in a sample size n is less than 0.01. Additionally, if every cell in a clonotype would be deleted, then the clonotype's exact subclonotype having the highest sum for umitot is found, summing across its cells. Then the cell in this exact subclonotype having the highest umitot value is saved from removal. The motivation for this filter is to mitigate illusory clonotype expansions arising from fragmentation of plasma cells or other physical processes. These processes all result in “cells” having low UMI counts, many of which do not correspond to intact real cells.

The third set of filters includes a twenty-second filter configured to remove some B cells having UMI counts below a threshold relative to UMI counts in a given clonotype. For example, a cell may be marked for removal if the V(D)J UMI count for some chain of some other cell is at least 500 times greater than the total V(D)J UMI count for the given cell. Then, each clonotype having at least two cells is scanned and every cell marked for removal is removed with the following qualification. Let k be the number of cells to be removed in a clonotype having n cells. Then, for a binomial distribution having p=0.1, it is required that the probability of observing k or more events in a sample size n is less than 0.01.

The third set of filters includes a twenty-third filter configured to remove cells called by the V(D)J pipeline of the Cell Ranger™ Software but not by the gene expression (GEX) pipeline of the Cell Ranger™ Software.

The third set of filters includes a twenty-fourth filter configured to remove some sequence barcode records that appear to represent doublets. For example, the twenty-fourth filter first computes pure subclonotypes by taking each clonotype and breaking it apart according to its chain signature, and all the exact subclonotypes that have entries for particular chains (and not entries for the other chains) are merged together to form a pure subclonotype. In the simplest case, where a clonotype has two chains, the clonotype could give rise to three pure subclonotypes: one for the exact subclonotypes that have both chains, and one each for the subclonotypes that have only one chain. The twenty-fourth filter finds triples (p0, p1, p2) of pure subclonotypes, for which the following three conditions are met: (1) p0 and p1 share an identical CDR3 DNA sequence, (2) p0 and p2 share an identical CDR3 DNA sequence, and (3) p1 and p2 do not share an identical CDR3 DNA sequence. The twenty-fourth filter then removes a pure subclonotype if five times the number of cells of a pure subclonotype (e.g., p0) is less than or equal to the smaller of the number of cells of the other pure subclonotypes (e.g., p1 and p2). After the twenty-fourth filter is applied, some of the original clonotypes may break up into separate clonotypes, as they may no longer be held together by shared chains.

The third set of filters includes a twenty-fifth filter configured to remove some sequence barcode records that appear to be contaminants based on the chain signature of the sequence barcode records. For example, the twenty-fifth filter sometimes breaks up complex clonotypes having many chains and representing multiple true clonotypes that are glued together into a single clonotype via exact subclonotypes whose constituent barcodes do not arise fully from single cells.

The third set of filters includes a twenty-sixth filter configured to prevent the merger of some single-chain clonotypes into other clonotypes. For example, the twenty-sixth filter may remove a clonotype or exact subclonotype having exactly one chain, and that are light chain or TRA gene, and whose number of cells is less than, for example, 0.1% of the total number of cells.

The third set of filters includes a twenty-seventh filter configured to remove cells having a chain that is likely spurious. For example, if a clonotype has three or more chains, and amongst those there is a chain that appears in a relatively small number of cells (e.g., less than a threshold), the twenty-seventh filter removes all the cells that support that chain.

The third set of filters includes a twenty-eighth filter configured to remove exact subclonotypes having a position with low quality scores. A Phred quality score (Q score) is a measure of the quality of the identification of the nucleobases generated by automated DNA sequencing.

As described above, the plurality of filters include a subset of filters that are used dependent upon whether the at least one data set includes GEX data. If the at least one data set does not include GEX data, then this subset of filters is used. Conversely, if the at least one data set does include GEX data, then this subset of filters is not used. In at least some embodiments, the GEX data dependent subset of filters includes the seventh, eighth, tenth, eleventh, fourteenth, twenty-first, and twenty-second filters described above. In general, these filters included in the GEX data dependent subset of filters are configured to remove a first sequence barcode record from the at least one data set including the first sequence barcode record and a second sequence barcode record where the first sequence barcode record comprises an identical V(D)J sequence as the second sequence barcode record. Using the filters in the GEX data dependent subset of filters when a data set includes gene expression data can result in a lower recovery of cells than expected from the data set because a majority of legitimate clonotypes that have a shared chain get filtered out. Accordingly, the GEX data dependent subset of filters are not used (e.g., not activated, deactivated, selected, etc.) when gene expression data is present in the at least one data set.

### Data Acquisition

In accordance with various embodiments, systems and methods within the disclosure include receiving a data set (e.g., a at least one data set). The at least one data set can be a sequence data set in which each sequence barcode record in the data set comprises at least one V(D)J sequence. The at least one data set can be a variable domain region sequence data set. The at least one data set can include a plurality of variable domain region sequences including both heavy chain region and light chain region sequences of antibodies and immunoglobulins, T-cell receptors (TCRs), or B-cell receptors (BCRs). The sequences in the at least one data set can represent the heavy chain variable region and light chain variable region sequences for each individual cell in a sample.

The B cell and T cell variable domain regions of the heavy and light chains contain multiple copies of V, J, and in some instances D gene segments for the variable regions of the antibody proteins. The variable domain region of the heavy chain contains V, D, and J gene segments, whereas the variable domain region of the light chain contains only V and J gene segments and lack a D gene segment. Accordingly, the B cell and/or T cell receptor dataset includes light chain sequences containing the V and J segments and heavy chain sequences containing the V, D and J segments.

The sample can be any biological sample, including for example, blood, tissue, cells, cell cultures, urine, or saliva. Another example of a sample can be a tube of cells from a donor or subject, from a particular tissue at a particular point in time, and possibly enriched for particular cells. The terms donor and subject are used interchangeably herein. A donor or a subject is an individual from which samples are obtained. The donor or subject can be a mammalian subject, including for example, a human, swine, monkey, ape, dog, cat, mouse (e.g., a humanized mouse), or rat. In some embodiments, the sample can be a splenocyte sample, a lymphocyte sample, or a bone marrow sample obtained from a mammalian subject.

The various embodiments, systems and methods within the disclosure further include receiving and processing sequence output data, for example, Chromium™ single-cell RNA-sequence output data. The Chromium™ single-cell RNA-sequencing technology takes samples containing cells of interests (e.g., a lymphoid cell such as a B cell or a T cell), and uses microfluidic partitioning to capture single cells in the sample and prepares uniquely barcoded, beads called Gel Bead-In Emulsions (GEMs), which are then used to derive barcoded cDNA libraries and sequenced by Illumina® sequencing instruments to generate the sequencing output data. As an example, a compatible format of the sequencing data can be as FASTQ files. One example of a software tool that processes and inputs the sequencing output data for producing the at least one data set within the disclosure can be the Cell Ranger™ Software. The Cell Ranger™ Software processes the Chromium single-cell RNA-sequence output data and transforms the sequencing output data into input dataset ready for analysis by the various embodiments, systems and methods within the disclosure. Accordingly, as an example within the disclosure, a data set can include all sequencing data obtained from a particular library type (e.g., TCR or BCR), from one cell group, processed by running thorough the Cell Ranger™ Software pipeline. It is understood that other software tools are also contemplated within the disclosure for processing and transforming the sequencing output data into input files.

### Cell Comparison and Grouping

In accordance with various embodiments, one or more comparison criteria can be utilized to sufficiently identify clonotypes or subclonotypes. These criteria need not be utilized as a group. It is understood that, certain criterion can be used independently or in combination with other steps discussed herein, while other criterion can only be used in combination with other steps discussed herein, in accordance with various embodiments within the disclosure. It is also understood that the criteria discussed below are simply examples, and not exhaustive. As such, the possible comparison criteria should not be limited to just those discussed herein. Comparison criteria can include, for example, computing the germline alleles for the donor's V segments, separately joining singletons, two libraries from the same tube of cells, same length V and J portion and predetermined threshold of nucleotide differences, same length CDRs and maximum number of nucleotide differences, same barcode with at least two cells, and comparison with the reference and shared mutations. For two cell clonotypes, comparison criteria can also include, for example, determining the number of CDR differences between the cell members, and determining that the comparison criteria is not met if the number of CDR differences exceed a determined two-cell threshold. Two-cell clonotype can be determined at least by cd≤d/2 (where cd is the number of differences between the given CDR3 nucleotide sequences and d are shared mutations between the two cells). In accordance with various embodiments, the two-cell threshold therefore can have a value dependent on the number of shared mutations.

Further, in accordance with various embodiments, various system and methods can further include identifying subclonotypes within an identified clonotype. The subclonotype includes cells having identical V(D)J transcripts. The subclonotype can further include cells having an identical C segment, same distance between a J stop codon and a C start codon, or both. The subclonotype can include cells having two or three chains.

### Computer System

FIG. 6 is a block diagram that illustrates a computer system 600, upon which embodiments of the present teachings may be implemented. In various embodiments of the present teachings, computer system 600 can include a bus 602 or other communication mechanism for communicating information, and a processor 604 coupled with bus 602 for processing information. In various embodiments, computer system 600 can also include a memory, which can be a random access memory (RAM) 606 or other dynamic storage device, coupled to bus 602 for determining instructions to be executed by processor 604. Memory also can be used for storing temporary variables or other intermediate information during execution of instructions to be executed by processor 604. In various embodiments, computer system 600 can further include a read only memory (ROM) 608 or other static storage device coupled to bus 602 for storing static information and instructions for processor 604. A storage device 610, such as a magnetic disk or optical disk, can be provided and coupled to bus 602 for storing information and instructions.

In various embodiments, computer system 600 can be coupled via bus 602 to a display 612, such as a cathode ray tube (CRT) or liquid crystal display (LCD), for displaying information to a computer user. An input device 614, including alphanumeric and other keys, can be coupled to bus 602 for communicating information and command selections to processor 604. Another type of user input device is a cursor control 616, such as a mouse, a trackball or cursor direction keys for communicating direction information and command selections to processor 604 and for controlling cursor movement on display 612. This input device 614 typically has two degrees of freedom in two axes, a first axis (i.e., x) and a second axis (i.e., y), that allows the device to specify positions in a plane. However, it should be understood that input devices 614 allowing for 3 dimensional (x, y and z) cursor movement are also contemplated herein.

Consistent with certain implementations of the present teachings, results can be provided by computer system 600 in response to processor 604 executing one or more sequences of one or more instructions contained in memory 606. Such instructions can be read into memory 606 from another computer-readable medium or computer-readable storage medium, such as storage device 610. Execution of the sequences of instructions contained in memory 606 can cause processor 604 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 604 for execution. Such a medium can take many forms, including but not limited to, non-volatile media, volatile media, and transmission media. Examples of non-volatile media can include, but are not limited to, optical, solid state, magnetic disks, such as storage device 610. Examples of volatile media can include, but are not limited to, dynamic memory, such as memory 606. Examples of transmission media can include, but are not limited to, coaxial cables, copper wire, and fiber optics, including the wires that comprise bus 602.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 604 of computer system 600 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 600 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 600 of Appendix D, whereby processor 604 would execute the analyses and determinations provided by these engines, subject to instructions provided by any one of, or a combination of, memory components 606/608/610 and user input provided via input device 614.

### Digital Processing Device

In various embodiments, the systems and methods described herein can include a digital processing device, or use of the same. In various embodiments, the digital processing device can includes one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeBSD, OpenBSD, Net-BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black-Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. In some embodiments, the non-volatile memory comprises dynamic random-access memory (DRAM). In various embodiments, the non-volatile memory comprises ferroelectric random access memory (FRAM). In various embodiments, the non-volatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active-matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion, or the like. In various embodiments, the input device is a combination of devices such as those disclosed herein.

### Non-Transitory Computer Readable Storage Medium

In various embodiments, and as stated above, the systems and methods disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

### Computer Program

In various embodiments, the systems and methods disclosed herein can include at least one computer program, or use at least one computer program. A computer program includes a sequence of instructions, executable in the digital processing device's CPU, written to perform a specified task. Computer readable instructions may be implemented as program modules, such as functions, objects, Application Programming Interfaces (APis), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft® NET or Ruby on Rails (RoR). In various embodiments, a web application utilizes one or more database systems including, by way of non-limiting examples, relational, non-relational, object oriented, associative, and XML database systems. In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client-side scripting languages, server-side coding languages, data-base query languages, or combinations thereof. In various embodiments, a web application is written to some extent in a markup language such as Hypertext Markup Language (HTML), Extensible Hypertext Markup Language (XHTML), or eXtensible Markup Language (XML). In various embodiments, a web application is written to some extent in a presentation definition language such as Cascading Style Sheets (CSS). In various embodiments, a web application is written to some extent in a client-side scripting language such as Asynchronous Javascript and XML (AJAX), Flash® Actionscript, Javascript, or Silverlight®. In various embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™ and Unity®.

### Mobile Application

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelera-tor®, Celsius, Bedrock, Flash Lite, NET Compact Frame-work, Rhomobile, and WorkLight Mobile Platform. Other development environments are available without cost including, by way of non-limiting examples, Lazarus, Mobi-Flex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo DSi Shop.

### Standalone Application

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often per-formed, at least in part, to create an executable program. In various embodiments, a computer program includes one or more executable complied applications.

### Web Browser Plug-in

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver-light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, and VB NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

### Software Modules

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules are hosted on one or more machines in one location. In various embodiments, software modules are hosted on one or more machines in more than one location.

### Databases

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object oriented databases, object databases, entity-relation-ship model databases, associative databases, and XML databases. Further non-limiting examples include SQL, Postgr-eSQL, MySQL, Oracle, DB2, and Sybase. In various embodiments, a database is internet-based. In further Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, a database is web-based. In various embodiments, a database is cloud computing-based. In other embodiments, a database is based on one or more local computer storage devices.

### Data Security

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In some instances, a user is locked out of an account after failing to input a proper username and password. The systems and methods disclosed herein can, in various embodiments, also include a mechanism for protecting the anonymity of users' genomes and of their searches across any genomes.

### Recitation of Embodiments

Embodiment 1: A computer-implemented method for analyzing nucleic acid sequences comprising:
receiving, at a computing device, at least one data set comprising a plurality of sequence barcode records from a plurality of single cells, wherein each sequence barcode record in the at least one data set comprises at least one V(D)J sequence;
selecting, with the computing device, one or more filters from a plurality of filters based on whether the at least one data set includes gene expression data, wherein
   if gene expression data is absent from the at least one data set, then a first set of filters is selected, wherein the first set of filters includes a second set of filters configured to remove a first sequence barcode record from the at least one data set including the first sequence barcode record and a second sequence barcode record where the first and second sequence barcode records comprise an identical V(D)J sequence, and
   if gene expression data is present in the at least one data set, then a third set of filters is selected, wherein the third set of filters includes the first set of filters minus the second set of filters; and
generating, with the computing device, a filtered data set that identifies one or more V(D)J cells from the plurality of single cells by filtering the at least one data set using the selected filters.

Embodiment 2: The method of Embodiment 1, wherein the at least one data set comprises a first data set and a second data set, and wherein the sequence barcode records of the second data set comprise a plurality of gene expression nucleic acid sequence barcode records such that gene expression data is present in the at least one data set.

Embodiment 3: The method of Embodiment 2, wherein each gene expression nucleic acid sequence barcode record comprises at least one cDNA sequence.

Embodiment 4: The method of Embodiment 2, wherein the sequence barcode records of the first data set are from the same plurality of single cells as the sequence barcode records of the second data set.

Embodiment 5: The method of Embodiment 2, further comprising generating, with the computing device, a modified second data set by determining sequence barcode records of the plurality of gene expression nucleic acid sequence barcode records in the second data set that are associated with a single cell of the plurality of single cells.

Embodiment 6: The method of Embodiment 5, wherein the plurality of gene expression nucleic acid sequence barcode records include data associated with a plurality of partitions wherein each partition comprises a single cell and data associated with partitions comprising two or more cells, and wherein generating the modified second data set comprises removing from the second data set the data associated with partitions comprising two or more cells.

Embodiment 7: The method of Embodiment 5, wherein the third set of filters includes a first subset of filters and a second subset of filters, and wherein generating the filtered data set comprises:
generating, with the computing device, a third data set by filtering the sequence barcode records of the first data set using the first subset of filters;
generating, with the computing device, a fourth data set by removing from the third data set any sequence barcode record in the third data set that is absent from the modified second data set; and
generating, with the computing device, the filtered data set by filtering the fourth data set using the second subset of filters.

Embodiment 8: The method of Embodiment 7, wherein the first subset of filters are configured to remove sequence barcode records from the first data set that have a transcript expression level below a threshold.

Embodiment 9: The method of Embodiment 7, wherein the second subset of filters comprises a plurality of filters configured to remove from the fourth data set sequence barcode records that contain material from two or more cells, cell fragments, or individual mRNA molecules.

Embodiment 10: The method of any one of the preceding Embodiments, further comprising generating, with the computing device, a modified filtered data set by grouping the one or more V(D)J cells in the filtered data set into at least one clonotype.

Embodiment 11: The method of Embodiment 10, further comprising: identifying, with the computing device and from the modified filtered data set, a clonotype having a quantity of chains greater than a predetermined threshold, the clonotype including a plurality of subclonotypes; and replacing, with the computing device, the identified clonotype with each of the plurality of subclonotypes such that each subclonotype becomes a respective clonotype.

Embodiment 12: The method of any one of the preceding Embodiments, wherein each of the one or more V(D)J cells are a T cell and/or a B cell.

Embodiment 13: The method of any one of the preceding Embodiments, further comprising detecting, with the computing device, whether the at least one data set includes gene expression data.

Embodiment 14: The method of any one of the preceding Embodiments, wherein the second set of filters includes a filter configured to remove a sequence barcode record based on a unique molecular identifier (UMI) count of the sequence barcode record.

Embodiment 15: A system comprising: a memory; and a processor in communication with the memory, the processor configured to perform the operations comprising: the method steps of any of Embodiments 1 to 14.

Embodiment 16: A non-transitory, computer-readable medium storing instructions, which when executed by a processor, cause the processor to perform the operations comprising: the method steps of any of Embodiments 1 to 14.

Embodiment 17: A computer-implemented method for analyzing nucleic acid sequences comprising:
receiving, at a computing device, a first data set and a second data set, each comprising a plurality of sequence barcode records from a plurality of single cells, wherein each sequence barcode record in the first and second data sets comprises at least one V(D)J sequence, and wherein the sequence barcode records of the second data set comprise a plurality of gene expression nucleic acid sequence barcode records that are associated with a single cell of the plurality of single cells;
generating, with the computing device, a third data set by filtering the first data set using a first set of filters configured to remove sequence barcode records from the first data set that have a transcript expression level below a threshold;
generating, with the computing device, a fourth data set by removing from the third data set any sequence barcode record in the third data set that is absent from the second data set; and
generating, with the computing device, a fifth data set that identifies one or more V(D)J cells from the plurality of single cells by filtering the fourth data set using a second set of filters configured to remove from the second filtered data set sequence barcode records that contain material from two or more cells, cell fragments, or individual mRNA molecules.

Embodiment 18: The method of Embodiment 17, wherein each gene expression nucleic acid sequence barcode record of the second data set comprises at least one cDNA sequence.

Embodiment 19: The method of any of Embodiments 17 or 18, further comprising generating a modified fifth data set by grouping the one or more V(D)J cells in the fifth data set into at least one clonotype.

Embodiment 20: The method of Embodiment 19, further comprising: identifying, from the modified fifth data set, a clonotype having a quantity of chains greater than a predetermined threshold, the clonotype including a plurality of subclonotypes; and replacing the identified clonotype with each of the plurality of subclonotypes such that each subclonotype becomes a respective clonotype.

Embodiment 21: The method of any of Embodiments 17 to 20, wherein the one or more V(D)J cells are a T cell and/or a B cell.

Embodiment 22: A system comprising: a memory; and a processor in communication with the memory, the processor configured to perform the operations comprising: the method steps of any of Embodiments 17 to 21.

Embodiment 23: A non-transitory, computer-readable medium storing instructions, which when executed by a processor, cause the processor to perform the operations comprising: the method steps of any of Embodiments 17 to 21.

Embodiment 24: A computer-implemented method for analyzing nucleic acid sequences comprising:
receiving, at a computing device, a data set comprising a plurality of sequence barcode records from a plurality of single cells, wherein each sequence barcode record in the data set comprises at least one V(D)J sequence;
generating, with the computing device, a first filtered data set by filtering the data set using a first set of filters configured to remove sequence barcode records from the data set that have a transcript expression level below a threshold;
generating, with the computing device, a second filtered data set by filtering the first filtered data set using a second set of filters configured to remove from the first filtered data set a clone that shares a chain with another clone; and
generating, with the computing device, a third filtered data set that identifies one or more V(D)J cells from the plurality of single cells by filtering the second filtered data set using a third set of filters configured to remove from the second filtered data set sequence barcode records that contain material from two or more cells, cell fragments, or individual mRNA molecules.

Embodiment 25: The method of Embodiment 24, further comprising generating a modified fifth data set by grouping the one or more V(D)J cells in the fifth data set into at least one clonotype.

Embodiment 26: The method of Embodiments 24 or 25, wherein the one or more V(D)J cells are a T cell and/or a B cell.

Embodiment 27: A system comprising: a memory; and a processor in communication with the memory, the processor configured to perform the operations comprising: the method steps of any of Embodiments 24 to 26.

Embodiment 28: A non-transitory, computer-readable medium storing instructions, which when executed by a processor, cause the processor to perform the operations comprising: the method steps of any of Embodiments 24 to 26.

Embodiment 29: A computer-implemented method for analyzing nucleic acid sequences comprising:
receiving, at a computing device, a first data set and a GEX cells data set, each comprising a plurality of sequence barcode records from a plurality of single cells, wherein each sequence barcode record in the first data set comprises at least one V(D)J sequence, and wherein the sequence barcode records of the GEX cells data set comprise a plurality of gene expression nucleic acid sequence barcode records that are identified as a single cell among the plurality of single cells;
generating, with the computing device, a second data set by filtering the first data set using a first set of filters;
generating, with the computing device, a third data (with GEX) set by removing from the second data set any sequence barcode record that is absent among the sequence barcode records of the GEX cells data set; and
generating, with a computing device, a fourth data set (with GEX) that comprise one or more V(D)J cells from the plurality of single cells by filtering the third data set using a fourth set of filters.

Embodiment 30: The method of Embodiment 29, wherein the first set of filters is configured to remove sequence barcode records from the first data set that have a transcript expression level below a threshold.

Embodiment 31: The method of Embodiment 29 or 30, wherein the fourth set of filters configured to remove from the third data set sequence barcode records that contain material from two or more cells.

Embodiment 32: The method of any one of Embodiments 29 to 31, wherein the plurality of sequence barcode records from the plurality of single cells are generated by partitioning a plurality of cells into single cell partitions.

Embodiment 33: The method any one of Embodiments 29 to 32, wherein at least one of the plurality of gene expression nucleic acid sequence barcode records in the GEX cells data set comprises at least one cDNA sequence.

Embodiment 34: The method of any of Embodiments 29 to 33, wherein the sequence barcode records of the first data set are from the same plurality of single cells as the sequence barcode records of the GEX cells data set.

Embodiment 35: The method of any of Embodiments 29 to 34, further comprising generating a fifth data (with GEX) set by grouping the one or more V(D)J cells in the fourth data set into at least one clonotype.

Embodiment 36: The method of any one of Embodiments 29 to 35, further comprising: identifying, from the fifth data set, a clonotype having a number/quantity of chains greater than a predetermined threshold, the clonotype including a plurality of subclonotypes; and replacing the identified clonotype with each of the plurality of subclonotypes such that each subclonotype becomes a respective/independent clonotype.

Embodiment 37: The method of any of Embodiments 29 to 36, wherein the one or more V(D)J cell is a T cell and/or a B cell.

Embodiment 38: The method of any one of Embodiments 29 to 37, wherein the first data set is generated from a V(D)J nucleic acid sequencing library, optionally wherein the first data set is generated by applying a first subset of filters to the V(D)J nucleic acid library.

Embodiment 39: The method of any one of Embodiments 29 to 38, wherein the GEX cells data set is generated from a GEX nucleic acid sequencing library from the plurality of single cells, optionally wherein the GEX cell data set is generated by applying a second subset of filters the GEX nucleic acid library.

Embodiment 40: A system comprising: a memory; and a processor in communication with the memory, the processor configured to perform the operations comprising: the method steps of any of Embodiments 29 to 39.

Embodiment 41: A non-transitory, computer-readable medium storing instructions, which when executed by a processor, cause the processor to perform the operations comprising: the method steps of any of Embodiments 29 to 39.

Embodiment 42: A computer-implemented method for analyzing nucleic acid sequences comprising:
receiving, at a computing device, at least one data set comprising a plurality of sequence barcode records from a plurality of single cells, wherein each sequence barcode record in the at least one data set comprises at least one V(D)J sequence;
selecting, with the computing device, one or more filters from a plurality of filters based on whether the at least one data set includes gene expression data, wherein
   if gene expression data is absent from the at least one data set, then a first set of filters is selected, wherein the first set of filters includes at least one filter configured to remove a first sequence barcode record from the at least one data set including the first sequence barcode record and a second sequence barcode record where the first and second sequence barcode records comprise an identical V(D)J sequence, and
   if gene expression data is present in the at least one data set, then a third set of filters is selected, wherein the third set of filters includes the first set of filters excluding the at least one filter; and
generating, with the computing device, a filtered data set that identifies one or more V(D)J cells from the plurality of single cells by filtering the at least one data set using the selected filters.

What is claimed:

1. A computer-implemented method for analyzing nucleic acid sequences comprising:
receiving, at a computing device, at least one data set comprising a plurality of sequence barcode records from a plurality of single cells, wherein each sequence barcode record in the at least one data set comprises at least one V(D)J sequence;
selecting, with the computing device, one or more filters from a plurality of filters based on whether the at least one data set includes gene expression data, wherein
if gene expression data is absent from the at least one data set, then a first set of filters is selected, wherein the first set of filters includes a second set of filters configured to remove a first sequence barcode record from the at least one data set including the first sequence barcode record and a second sequence barcode record where the first and second sequence barcode records comprise an identical V(D)J sequence, and
if gene expression data is present in the at least one data set, then a third set of filters is selected, wherein the third set of filters includes the first set of filters minus the second set of filters; and
generating, with the computing device, a filtered data set that identifies one or more V(D)J cells from the plurality of single cells by filtering the at least one data set using the selected filters.

2. The method of claim 1, wherein the at least one data set comprises a first data set and a second data set, and wherein the sequence barcode records of the second data set comprise a plurality of gene expression nucleic acid sequence barcode records such that gene expression data is present in the at least one data set.

3. The method of claim 2, wherein each gene expression nucleic acid sequence barcode record comprises at least one cDNA sequence.

4. The method of claim 2, wherein the sequence barcode records of the first data set are from the same plurality of single cells as the sequence barcode records of the second data set.

5. The method of claim 2, further comprising generating, with the computing device, a modified second data set by determining sequence barcode records of the plurality of gene expression nucleic acid sequence barcode records in the second data set that are associated with a single cell of the plurality of single cells.

6. The method of claim 5, wherein the plurality of gene expression nucleic acid sequence barcode records include data associated with a plurality of partitions wherein each partition comprises a single cell and data associated with partitions comprising two or more cells, and wherein generating the modified second data set comprises removing from the second data set the data associated with partitions comprising two or more cells.

7. The method of claim 5, wherein the third set of filters includes a first subset of filters and a second subset of filters, and wherein generating the filtered data set comprises:
generating, with the computing device, a third data set by filtering the sequence barcode records of the first data set using the first subset of filters;
generating, with the computing device, a fourth data set by removing from the third data set any sequence barcode record in the third data set that is absent from the modified second data set; and
generating, with the computing device, the filtered data set by filtering the fourth data set using the second subset of filters.

8. The method of claim 7, wherein the first subset of filters are configured to remove sequence barcode records from the first data set that have a transcript expression level below a threshold.

9. The method of claim 7, wherein the second subset of filters comprises a plurality of filters configured to remove from the fourth data set sequence barcode records that contain material from two or more cells, cell fragments, or individual mRNA molecules.

10. The method of claim 1, further comprising generating, with the computing device, a modified filtered data set by grouping the one or more V(D)J cells in the filtered data set into at least one clonotype.

11. The method of claim 10, further comprising:
identifying, with the computing device and from the modified filtered data set, a clonotype having a quantity of chains greater than a predetermined threshold, the clonotype including a plurality of subclonotypes; and
replacing, with the computing device, the identified clonotype with each of the plurality of subclonotypes such that each subclonotype becomes a respective clonotype.

12. The method of claim 1, wherein the second set of filters includes a filter configured to remove a sequence barcode record based on a unique molecular identifier (UMI) count of the sequence barcode record.

13. A computer-implemented method for analyzing nucleic acid sequences comprising:
receiving, at a computing device, a first data set and a second data set, each comprising a plurality of sequence barcode records from a plurality of single cells, wherein each sequence barcode record in the first and second data sets comprises at least one V(D)J sequence, and wherein the sequence barcode records of the second data set comprise a plurality of gene expression nucleic acid sequence barcode records that are associated with a single cell of the plurality of single cells;
generating, with the computing device, a third data set by filtering the first data set using a first set of filters configured to remove sequence barcode records from the first data set that have a transcript expression level below a threshold;
generating, with the computing device, a fourth data set by removing from the third data set any sequence barcode record in the third data set that is absent from the second data set; and
generating, with the computing device, a fifth data set that identifies one or more V(D)J cells from the plurality of single cells by filtering the fourth data set using a second set of filters configured to remove from the second filtered data set sequence barcode records that contain material from two or more cells, cell fragments, or individual mRNA molecules.

14. The method of claim 13, wherein each gene expression nucleic acid sequence barcode record of the second data set comprises at least one cDNA sequence.

15. The method of claim 13, further comprising generating a modified fifth data set by grouping the one or more V(D)J cells in the fifth data set into at least one clonotype.

16. The method of claim 15, further comprising:
identifying, from the modified fifth data set, a clonotype having a quantity of chains greater than a predetermined threshold, the clonotype including a plurality of subclonotypes; and
replacing the identified clonotype with each of the plurality of subclonotypes such that each subclonotype becomes a respective clonotype.

17. The method of claim 1, wherein the one or more V(D)J cells are a T cell and/or a B cell.

18. A computer-implemented method for analyzing nucleic acid sequences comprising:
receiving, at a computing device, a data set comprising a plurality of sequence barcode records from a plurality of single cells, wherein each sequence barcode record in the data set comprises at least one V(D)J sequence;
generating, with the computing device, a first filtered data set by filtering the data set using a first set of filters configured to remove sequence barcode records from the data set that have a transcript expression level below a threshold;
generating, with the computing device, a second filtered data set by filtering the first filtered data set using a second set of filters configured to remove from the first filtered data set a clone that shares a chain with another clone; and
generating, with the computing device, a third filtered data set that identifies one or more V(D)J cells from the plurality of single cells by filtering the second filtered data set using a third set of filters configured to remove from the second filtered data set sequence barcode records that contain material from two or more cells, cell fragments, or individual mRNA molecules.

19. The method of claim 18, further comprising generating a modified fifth data set by grouping the one or more V(D)J cells in the fifth data set into at least one clonotype.

20. The method of claim 18, wherein the one or more V(D)J cells are a T cell and/or a B cell.
